(19) United States
(12) Patent Application Publication
(10) Pub. No.: US 2024/0408354 A1
(43) Pub. Date: Dec. 12, 2024

(54) MEDICAL APPARATUS

(71) Applicant: CANON KABUSHIKI KAISHA, Tokyo (JP)

(72) Inventors: DAISUKE KANEKO, Tokyo (JP); HIROAKI OHASHI, Tokyo (JP); YUSUKE NIIKAWA, Kanagawa (JP); TOMIO NOGUCHI, Shizuoka (JP)

(21) Appl. No.: 18/812,144

(22) Filed: Aug. 22, 2024

Related U.S. Application Data
(63) Continuation of application No. PCT/JP2023/003499, filed on Feb. 3, 2023.

(30) Foreign Application Priority Data
Feb. 22, 2022 (JP) 2022-025372

(51) Int. Cl. A61M 25/01

(52) U.S. Cl. CPC A61M 25/01; A61M 2205/10; A61M 2205/50

(57) ABSTRACT

A medical apparatus includes (i) a coupling unit including a coupling portion connected to a driving source, (ii) a bending unit including a linear member connected to a bending portion, (iii) a first operation portion movable to a fixation position in which the linear member is fixed to the coupling portion and to an attachment-detachment position, (iv) a lock member movable to a lock position in which the lock member restricts detachment of the bending unit from the coupling unit and to an unlock position, and (v) a second operation portion that moves the lock member to the unlock position. Detachment of the bending unit from the coupling unit is allowed in a state in which the bending unit is attached to the coupling unit, in which the first operation portion is in the attachment-detachment position, and in which the lock member is in the unlock position.

### CROSS-REFERENCE TO RELATED APPLICATIONS

This application is a Continuation of International Patent Application No. PCT/JP2023/003499, filed Feb. 3, 2023, which claims the benefit of Japanese Patent Application No. 2022-025372, filed Feb. 22, 2022, both of which are hereby incorporated by reference herein in their entirety.

### TECHNICAL FIELD

The present invention relates to a medical apparatus that includes a linear member that bends a bending portion.

### BACKGROUND ART

U.S. Patent Application Publication No. 2021/0121051 discloses a medical apparatus in which a coupling shaft including a wire that bends a bending portion and a rod that is connected to the wire is detachably attached to a coupling receiver that includes a driving stage connected to an actuator.

In the medical apparatus disclosed in U.S. Patent Application Publication No. 2021/0121051, the coupling shaft is linearly inserted in one direction into the coupling receiver and is then rotated to thereby engage the rod with the driving stage and couple the wire to the actuator.

To disconnect the wire and the actuator from each other, the coupling shaft is rotated to disengage the rod and the driving stage from each other. A position in which the rod and the driving stage are disengaged from each other is also a position in which the coupling shaft is detachable from the coupling receiver.

When a bending unit that includes a linear member connected to a bending portion is detachable from a coupling unit that includes a coupling portion coupled to a driving source, it is preferable to suppress detachment of the bending unit from the coupling unit without an intention of a user.

### CITATION LIST

### Patent Literature

PTL 1: U.S. Patent Application Publication No. 2021/0121051

### SUMMARY OF INVENTION

An object of the present invention is to suppress a situation in which an operation for releasing a linear member from a coupling portion causes a bonding unit to be detachable from a coupling unit.

One aspect of the invention according to the present application is as follows.

A medical apparatus comprising:
a coupling unit that includes a plurality of coupling portions, the plurality of coupling portions being each connected to a corresponding one of a plurality of driving sources;
a bending unit that is detachably attached to the coupling unit, the bending unit including a bending portion and a plurality of linear members that are connected to the bending portion;
a first operation portion that is movable to a fixation position and to an attachment-detachment position in a state in which the bending unit is attached to the coupling unit, the fixation position being a position in which each of the plurality of linear members is fixed to a corresponding one of the plurality of coupling portions, the attachment-detachment position being a position in which each of the plurality of linear members is released from the corresponding one of the plurality of coupling portions;
a lock member that is movable to a lock position and to an unlock position in a state in which the bending unit is attached to the coupling unit and in which the first operation portion is in the attachment-detachment position, the lock position being a position in which the lock member restricts detachment of the bending unit from the coupling unit; and
a second operation portion that moves the lock member from the lock position to the unlock position,
wherein detachment of the bending unit from the coupling unit is allowed in a state in which the bending unit is attached to the coupling unit, in which the first operation portion is in the attachment-detachment position, and in which the lock member is in the unlock position, and
wherein the first operation portion is movable between the fixation position and the attachment-detachment position in a state in which the bending unit is attached to the coupling unit and in which the lock member is in the lock position.

One aspect of the invention according to the present application is as follows.

A medical apparatus comprising:
a coupling unit that includes a plurality of coupling portions, the plurality of coupling portions being each connected to a corresponding one of a plurality of driving sources;
a bending unit that is detachably attached to the coupling unit, the bending unit including a bending portion and a plurality of linear members that are connected to the bending portion;
a first operation portion that is movable to a fixation position and to an attachment-detachment position in a state in which the bending unit is attached to the coupling unit, the fixation position being a position in which each of the plurality of linear members is fixed to a corresponding one of the plurality of coupling portions, the attachment-detachment position being a position in which each of the plurality of linear members is released from the corresponding one of the plurality of coupling portions;
a lock member that is movable to a lock position and to an unlock position in a state in which the bending unit is attached to the coupling unit and in which the first operation portion is in the attachment-detachment position, the lock position being a position in which the lock member restricts detachment of the bending unit from the coupling unit; and
a second operation portion that moves the lock member from the lock position to the unlock position,
wherein detachment of the bending unit from the coupling unit is allowed in a state in which the bending unit is attached to the coupling unit, in which the first operation portion is in the attachment-detachment position, and in which the lock member is in the unlock position, and
wherein the second operation portion is positioned away from the first operation portion.

Further features of the present invention will become apparent from the following description of exemplary embodiments with reference to the attached drawings.

### BRIEF DESCRIPTION OF DRAWINGS

FIG. 1 is a general view of a medical system.

FIG. 2 is a perspective view illustrating a medical apparatus and a support base.

FIG. 3A is an explanatory view of a catheter.

FIG. 3B is an explanatory view of a catheter.

FIG. 4A is an explanatory view of a catheter unit.

FIG. 4B is an explanatory view of a catheter unit.

FIG. 5A is an explanatory view of a base unit and a wire drive portion.

FIG. 5B is an explanatory view of a base unit and a wire drive portion.

FIG. 5C is an explanatory view of a base unit and a wire drive portion.

FIG. 6A is an explanatory view of a wire drive portion, a coupling device, and a bend drive portion.

FIG. 6B is an explanatory view of a wire drive portion, a coupling device, and a bend drive portion.

FIG. 6C is an explanatory view of a wire drive portion, a coupling device, and a bend drive portion.

FIG. 7A is an explanatory view of attachment of a catheter unit.

FIG. 7B is an explanatory view of attachment of a catheter unit.

FIG. 8A is an exploded perspective view illustrating a first operation portion and components at the periphery of the first operation portion.

FIG. 8B is an exploded perspective view illustrating a first operation portion and components at the periphery of the first operation portion.

FIG. 9A is a view for describing a relationship among a first operation portion, a lock member, and a second operation portion.

FIG. 9B is a view for describing a relationship among a first operation portion, a lock member, and a second operation portion.

FIG. 10A is a view for describing a relationship among a first operation portion, a lock member, and a second operation portion.

FIG. 10B is a view for describing a relationship among a first operation portion, a lock member, and a second operation portion.

FIG. 11 is a sectional view of a catheter unit and a base unit.

FIG. 12 is a sectional view of a catheter unit and a base unit.

FIG. 13A is a sectional view of a catheter unit and a base unit.

FIG. 13B is a sectional view of a catheter unit and a base unit.

FIG. 14 is a sectional view of a catheter unit and a base unit.

FIG. 15 is a view for describing a catheter unit and a coupling portion of a base unit.

FIG. 16 is a view for describing fixation of a driving wire by a coupling portion.

FIG. 17 is a view for describing fixation of a driving wire by a coupling portion.

FIG. 18 is a view for describing fixation of a driving wire by a coupling portion.

FIG. 19 is a view for describing fixation of a driving wire by a coupling portion.

FIG. 20 is a view for describing fixation of a driving wire by a coupling portion.

### DESCRIPTION OF EMBODIMENTS

Hereinafter, a configuration of the present invention will be presented as an example with reference to the drawings. Note that dimensions, materials, shapes, arrangements, and the like of components described in the present embodiment should be changed, as appropriate, in accordance with a configuration, various conditions, and the like of an apparatus to which the present invention is applied.

### Embodiment 1

### Medical System and Medical Apparatus

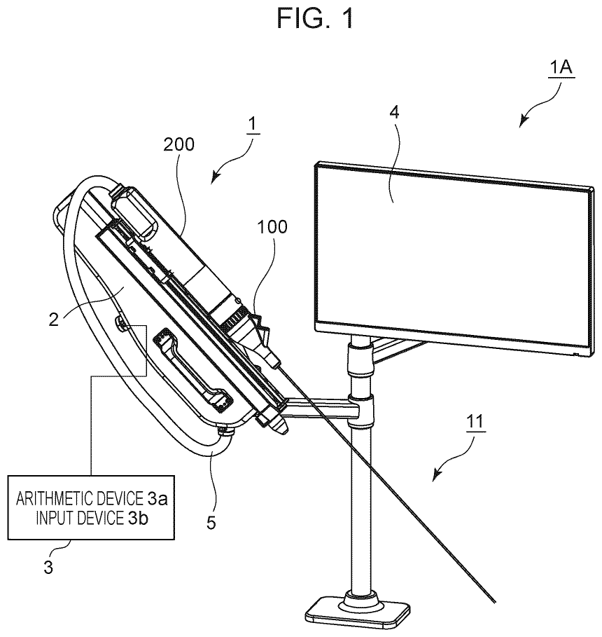

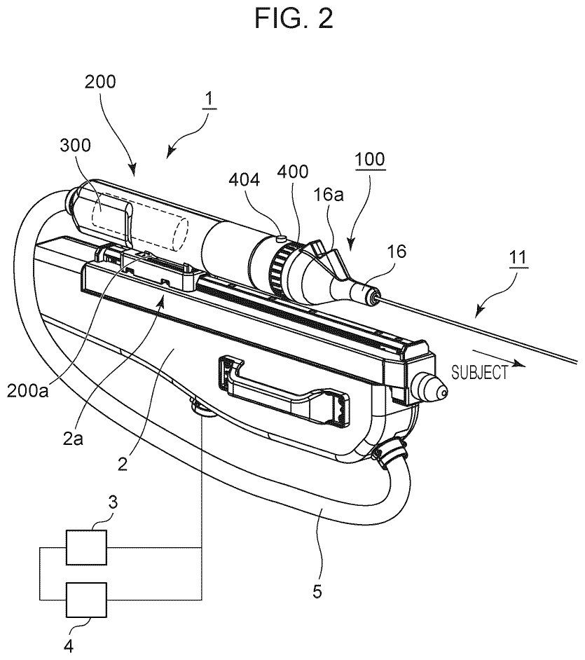

A medical system 1A and a medical apparatus 1 will be described with reference to FIG. 1 and FIG. 2. FIG. 1 is a general view of the medical system 1A. FIG. 2 is a perspective view illustrating the medical apparatus 1 and a support base 2.

The medical system 1A includes the medical apparatus 1, the support base 2 to which the medical apparatus 1 is to be attached, and a controller 3 that controls the medical apparatus 1. In the present embodiment, the medical system 1A includes a monitor 4 as a display device.

The medical apparatus 1 includes a catheter unit (bendable unit, bending unit, or bending-body unit) 100 that includes a catheter 11 as a bendable body, and a base unit (drive unit, attached unit, or coupling unit) 200. The catheter unit 100 is configured to be attachable to and detachable from the base unit 200.

In the present embodiment, a user of the medical system 1A and the medical apparatus 1 can perform operations, such as observation of the inside of a subject, collection of various specimens from the inside of the subject, and treatment with respect to the inside of the subject, by inserting the catheter 11 into the inside of the subject. In one embodiment, the user can insert the catheter 11 into the inside of a patient as the subject. Specifically, operations, such as observation, collection, resection, and the like of a lung tissue, can be performed by inserting the catheter 11 into a bronchial tube through an oral cavity or a nasal cavity of the patient.

The catheter 11 is usable as a guide (sheath) that guides a medical instrument for performing the aforementioned operations. Examples of the medical instrument (tool) are an endoscope, forceps, an ablation device, and the like. The catheter 11 itself may have a function as the aforementioned medical instrument.

In the present embodiment, the controller 3 includes an arithmetic device 3a and an input device 3b. The input device 3b receives a command or an input for operating the catheter 11. The arithmetic device 3a includes a storage in which a program and various data for controlling the catheter 11 are stored, a random-access memory, and a central processing unit for executing a program. The controller 3 may include an output unit that outputs a signal for displaying an image on the monitor 4.

As illustrated in FIG. 2, the medical apparatus 1 in the present embodiment is electrically connected to the controller 3 through a cable 5 that couples the base unit 200 of the medical apparatus 1 and the support base 2 to each other and the support base 2. The medical apparatus 1 and the controller 3 may be directly connected to each other through a cable. The medical apparatus 1 and the controller 3 may be connected to each other wirelessly.

The medical apparatus 1 is detachably attached to the support base 2 via the base unit 200. More specifically, an attachment portion (connection portion) 200a of the base unit 200 of the medical apparatus 1 is detachably attached to a moving stage (receiver) 2a of the support base 2. Connection between the medical apparatus 1 and the controller 3 is maintained, even in a state in which the attachment portion 200a of the medical apparatus 1 is detached from the moving stage 2a, to enable the controller 3 to control the medical apparatus 1. In the present embodiment, the medical apparatus 1 and the support base 2 are connected to each other by the cable 5, even in a state in which the attachment portion 200a of the medical apparatus 1 is detached from the moving stage 2a.

A user can insert the catheter 11 into the inside of a subject by moving the medical apparatus 1 manually in a state (state in which the medical apparatus 1 is detached from the moving stage 2a) in which the medical apparatus 1 is detached from the support base 2.

A user can use the medical apparatus 1 in a state in which the catheter 11 is inserted into a subject and in which the medical apparatus 1 is attached to the support base 2. Specifically, the medical apparatus 1 is moved by a movement of the moving stage 2a in a state in which the medical apparatus 1 is attached to the moving stage 2a. Then, an operation of moving the catheter 11 in a direction of insertion into a subject and an operation of moving the catheter 11 in a direction of extraction from the subject are performed. The movement of the moving stage 2a is controlled by the controller 3.

The medical apparatus 1 includes a wire drive portion (linear-member drive portion, line drive portion, or body drive portion) 300 for driving the catheter 11. In the present embodiment, the medical apparatus 1 is a robot catheter apparatus that drives the catheter 11 by the wire drive portion 300 controlled by the controller 3.

The controller 3 can perform an operation of bending the catheter 11 by controlling the wire drive portion 300. In the present embodiment, the wire drive portion 300 is incorporated in the base unit 200. More specifically, the base unit 200 includes a base housing 200f that houses the wire drive portion 300. That is, the base unit 200 includes the wire drive portion 300. The wire drive portion 300 and the base unit 200 can be collectively referred to as a catheter driving device (base device or body).

In an extension direction of the catheter 11, an end portion at which the leading end of the catheter 11, which is to be inserted into a subject, is disposed is referred to as the distal end. In the extension direction of the catheter 11, a side opposite to the distal end is referred to as the proximal end.

The catheter unit 100 includes a proximal end cover 16 that covers the proximal end side of the catheter 11. The proximal end cover 16 has a tool hole 16a. A medical instrument is insertable into the catheter 11 through the tool hole 16a.

As described above, the catheter 11 in the present embodiment has a function as a guide device for guiding a medical instrument to a desired position in the inside of a subject.

For example, the catheter 11 is inserted into a subject to a target position in a state in which an endoscope is inserted into the catheter 11. At this time, at least one of manual operation by a user, movement of the moving stage 2a, and driving of the catheter 11 by the wire drive portion 300 is used. After the catheter 11 reaches the target position, the endoscope is extracted from the catheter 11 through the tool hole 16a. Then, a medical instrument is inserted through the tool hole 16a, and operations, such as collection of various specimens from the inside of the subject and treatment with respect to the inside of the subject, are performed.

As described later, the catheter unit 100 is detachably attached to the catheter driving device (base device or body), more specifically, to the base unit 200. After using the medical apparatus 1, a user detaches the catheter unit 100 from the base unit 200 and can use the medical apparatus 1 again by attaching a new catheter unit 100 to the base unit 200.

As illustrated in FIG. 2, the medical apparatus 1 includes a first operation portion 400. In the present embodiment, the first operation portion 400 is included in the catheter unit 100. The first operation portion 400 is operated by a user to couple the catheter 11 and the wire drive portion 300 to each other and to disconnect the catheter 11 and the wire drive portion 300 from each other.

### Catheter

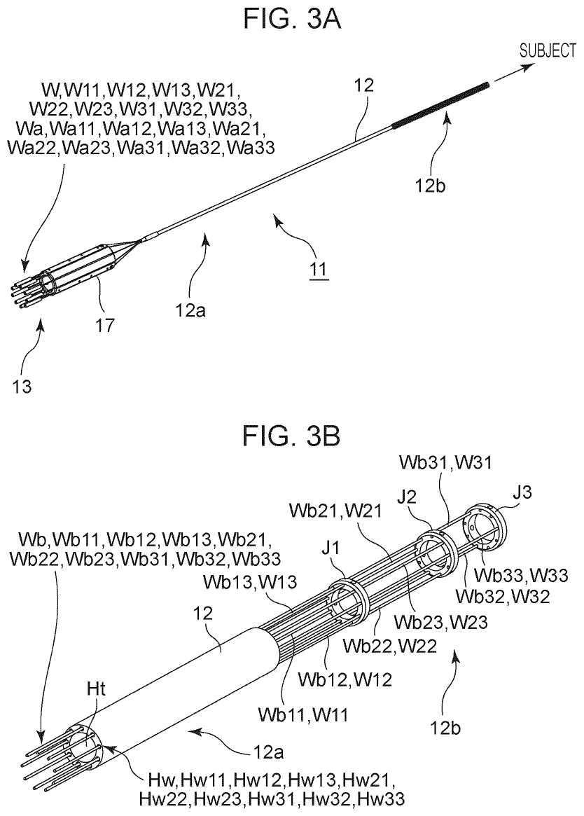

The catheter 11 as a bendable body will be described with reference to FIG. 3A and FIG. 3B. FIG. 3A and FIG. 3B are explanatory views of the catheter 11. FIG. 3A is a view for describing the entirety of the catheter 11. FIG. 3B is an enlarged view of the catheter 11.

The catheter 11 includes a bending portion (bending body or catheter body) 12 and a bend drive portion (catheter drive portion) 13 that is configured to bend the bending portion 12. The bend drive portion 13 is configured to bend the bending portion 12 by receiving a driving force of the wire drive portion 300 through a coupling device 21, which is described later.

The catheter 11 extends in a direction of insertion of the catheter 11 into a subject. The extension direction (longitudinal direction) of the catheter 11 is identical to the extension direction (longitudinal direction) of the bending portion 12 and the extension direction (longitudinal direction) of each of first to ninth driving wires (W11 to W33), which are described later.

The bend drive portion 13 includes a plurality of driving wires (driving lines, linear members, or linear actuators) that are connected to the bending portion 12. Specifically, the bend drive portion 13 includes a first driving wire W11, a second driving wire W12, a third driving wire W13, a fourth driving wire W21, a fifth driving wire W22, a sixth driving wire W23, a seventh driving wire W31, an eighth driving wire W32, and a ninth driving wire W33.

Each of the first to ninth driving wires (W11 to W33) includes a held portion (held shaft or rod) Wa. Specifically, the first driving wire W11 includes a first held portion Wa11. The second driving wire W12 includes a second held portion Wa12. The third driving wire W13 includes a third held portion Wa13. The fourth driving wire W21 includes a fourth held portion Wa21. The fifth driving wire W22 includes a fifth held portion Wa22. The sixth driving wire W23 includes a sixth held portion Wa23. The seventh driving wire W31 includes a seventh held portion Wa31. The eighth driving wire W32 includes an eighth held portion Wa32. The ninth driving wire W33 includes a ninth held portion Wa33.

In the present embodiment, the first to ninth held portions (Wa11 to Wa33) have identical shapes.

Each of the first to ninth driving wires (W11 to W33) includes a wire body (line body or linear body) Wb having flexibility. Specifically, the first driving wire W11 includes a first wire body Wb11. The second driving wire W12 includes a second wire body Wb12. The third driving wire W13 includes a third wire body Wb13. The fourth driving wire W21 includes a fourth wire body Wb21. The fifth driving wire W22 includes a fifth wire body Wb22. The sixth driving wire W23 includes a six wire body Wb23. The seventh driving wire W31 includes a seventh wire body Wb31. The eighth driving wire W32 includes an eighth wire body Wb32. The ninth driving wire W33 includes a ninth wire body Wb33.

In the present embodiment, the first to third wire bodies (Wb11 to Wb13) have identical shapes. The fourth to sixth wire bodies (Wb21 to Wb23) have identical shapes. The seventh to ninth wire bodies (Wb31 to Wb33) have identical shapes. In the present embodiment, the first to ninth wire bodies (Wb11 to Wb33) have identical shapes except for lengths.

The first to ninth held portions (Wa11 to Wa33) are each fixed at the proximal end of a corresponding one of the first to ninth wire bodies (Wb11 to Wb33) to the corresponding one of the first to ninth wire bodies (Wb11 to Wb33).

The first to ninth driving wires (W11 to W33) are inserted into and fixed in the bending portion 12 through a wire guide 17.

In the present embodiment, the material of each of the first to ninth driving wires (W11 to W33) is a metal. The material of each of the first to ninth driving wires (W11 to W33), however, may be a resin. The material of each of the first to ninth driving wires (W11 to W33) may contain a metal and a resin.

Any one of the first to ninth driving wires (W11 to W33) can be referred to as the driving wire W. In the present embodiment, the first to ninth driving wires (W11 to W33) have identical shapes except for lengths of the first to ninth wire bodies (Wb11 to Wb33).

In the present embodiment, the bending portion 12 is a tubular member that has flexibility and that includes a passage Ht for insertion of a medical instrument.

The bending portion 12 has a wall surface that has a plurality of wire holes through each of which a corresponding one of the first to ninth driving wires (W11 to W33) passes. Specifically, the wall surface of the bending portion 12 has a first wire hole Hw11, a second wire hole Hw12, a third wire hold Hw13, a fourth wire hole Hw21, a fifth wire hole Hw22, a sixth wire hole Hw23, a seventh wire hole Hw31, an eighth wire hole Hw32, and a ninth wire hole Hw33. The first to ninth wire holes Hw (Hw11 to Hw33) correspond to the first to ninth driving wires (W11 to W33), respectively. A number after the sign Hw indicates a number of a corresponding driving wire. For example, the first driving wire W11 is inserted into the first wire hole Hw11.

Any one of the first to ninth wire holes (Hw11 to Hw33) can be referred to as the wire hole Hw. In the present embodiment, the first to ninth wire holes (Hw11 to Hw33) have identical shapes.

The bending portion 12 includes an intermediate region 12a and a bending region 12b. The bending region 12b is disposed at the distal end of the bending portion 12, and a first guide ring J1, a second guide ring J2, and a third guide ring J3 are disposed in the bending region 12b. The bending region 12b is a region in which the degree and direction of bending of the bending portion 12 can be controlled by moving the first guide ring J1, the second guide ring J2, and the third guide ring J3 by the bend drive portion 13. In FIG. 3B, part of the bending portion 12 that covers the first to third guide rings (J1 to J3) is omitted.

In the present embodiment, the bending portion 12 includes a plurality of auxiliary rings (not illustrated). In the bending region 12b, the first guide ring J1, the second guide ring J2, and the third guide ring J3 are fixed to the wall surface of the bending portion 12. In the present embodiment, the plurality of auxiliary rings are disposed between the first guide ring J1 and the second guide ring J2 and between the second guide ring J2 and the third guide ring J3.

A medical instrument is guided to the leading end of the catheter 11 by the passage Ht, the first to third guide rings (J1 to J3), and the plurality of auxiliary rings.

The first to ninth driving wires (W11 to W33) pass through the intermediate region 12a and are fixed to the first to third guide rings (J1 to J3) corresponding thereto.

Specifically, the first driving wire W11, the second driving wire W12, and the third driving wire W13 are fixed to the first guide ring J1. The fourth driving wire W21, the fifth driving wire W22, and the sixth driving wire W23 extend through the first guide ring J1 and a plurality of the auxiliary rings and are fixed to the second guide ring J2. The seventh driving wire W31, the eighth driving wire W32, and the ninth driving wire W33 extend through the first guide ring J1, the second guide ring J2, and the plurality of auxiliary rings and are fixed to the third guide ring J3.

The medical apparatus 1 can bend the bending portion 12 in a direction intersecting the extension direction of the catheter 11 by driving the bend drive portion 13 by the wire drive portion 300. Specifically, it is possible by moving each of the first to ninth driving wires (W11 to W33) in the extension direction of the bending portion 12 to bend the bending region 12b of the bending portion 12 via the first to third guide rings (J1 to J3) in the direction intersecting the extension direction.

A user can insert the catheter 11 into a subject to a target portion by using at least one of manual movement of the medical apparatus 1, movement of the medical apparatus 1 by the moving stage 2a, and bending of the bending portion 12.

In the present embodiment, the first to third guide rings (J1 to J3) are moved by the first to ninth driving wires (W11 to W33), thereby bending the bending portion 12. The present invention is, however, not limited to this configuration. Any one or two of the first to third guide rings (J1 to J3) and the driving wires fixed thereto may be omitted.

For example, the catheter 11 may have a configuration in which the seventh to ninth driving wires (W31 to W33) and the third guide ring J3 are included and the first to sixth driving wires (W11 to W23) and the first and second guide rings (J1 and J2) are omitted. The catheter 11 may have a configuration in which the fourth to ninth driving wires (W21 to W33) and the second and third guide rings (J2 and J3) are included and the first to third driving wires (W11 to W13) and the first guide ring J1 are omitted.

The catheter 11 may have a configuration in which one guide ring is driven by two driving wires. Even in this case, the number of the guide rings may be one or more than one.

### Catheter Unit

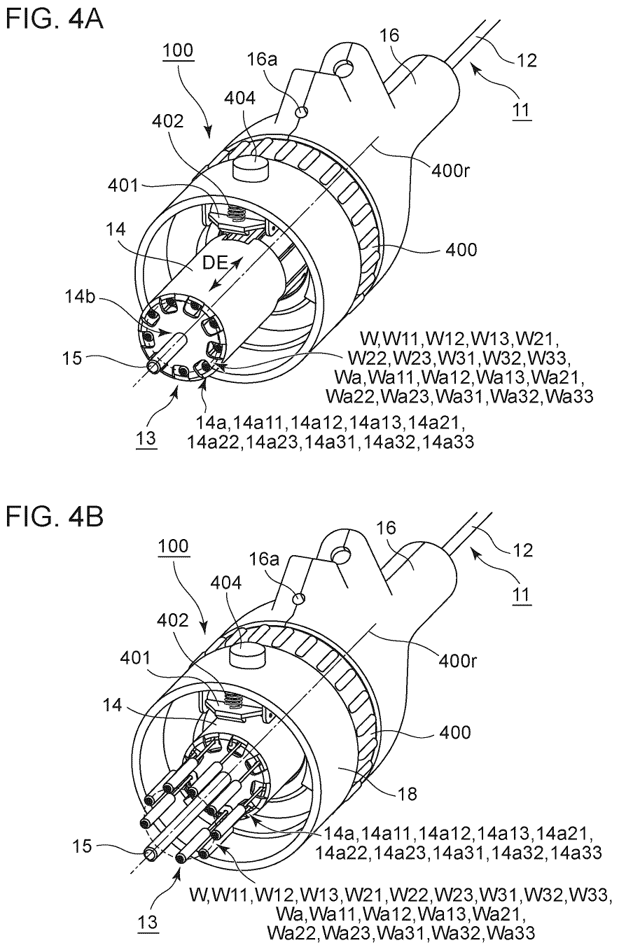

The catheter unit 100 will be described with reference to FIG. 4A and FIG. 4B.

FIG. 4A and FIG. 4B are explanatory views of the catheter unit 100. FIG. 4A is an explanatory view of the catheter unit 100 in a state in which a wire cover 14, which is described later, is in a cover position. FIG. 4B is an explanatory view of the catheter unit 100 in a state in which the wire cover 14, which is described later, is in an exposure position.

The catheter unit 100 includes the bending portion 12, the catheter 11 including the bend drive portion 13, and the proximal end cover 16 supporting the proximal end of the catheter 11. The catheter unit 100 includes a cover (wire cover) 14 for covering and protecting the first to ninth driving wires (W11 to W33) as the plurality of driving wires.

The catheter unit 100 is attachable to and detachable from the base unit 200 in an attachment-detachment direction DE. An attachment direction of the catheter unit 100 with respect to the base unit 200 and a detachment direction of the catheter unit 100 from the base unit 200 are parallel to the attachment-detachment direction DE.

The proximal end cover (frame body, bending-portion housing, or catheter housing) 16 is a cover that covers part of the catheter 11. The proximal end cover 16 has the tool hole 16a for insertion of a medical instrument into the passage Ht of the bending portion 12.

The wire cover 14 has a plurality of exposure holes (wire-cover holes or cover holes) through each of which a corresponding one of the first to ninth driving wires (W11 to W33) passes. The wire cover 14 has a first exposure hole 14a11, a second exposure hole 14a12, a third exposure hole 14a13, a fourth exposure hole 14a21, a fifth exposure hole 14a22, a sixth exposure hole 14a23, a seventh exposure hole 14a31, an eighth exposure hole 14a32, and a ninth exposure hole 14a33. The first to ninth exposure holes (14a11 to 14a33) correspond to the first to ninth driving wires (W11 to W33), respectively. A number after the sign 14a indicates a number of a corresponding driving wire. For example, the first driving wire W11 is inserted into the first exposure hole 14a11.

Any one of the first to ninth exposure holes (14a11 to 14a33) can be referred to as the exposure hole 14a. In the present embodiment, the first to ninth exposure holes (14a11 to 14a33) have identical shapes.

The wire cover 14 is movable to the cover position (refer to FIG. 4(a)) in which the wire cover 14 covers the first to ninth driving wires (W11 to W33) and to a cover retracted position (refer to FIG. 4(b)) in which the wire cover 14 is retracted from the cover position. The cover retracted position can be referred to as an exposure position in which the first to ninth driving wires (W11 to W33) are exposed.

Before the catheter unit 100 is attached to the base unit 200, the wire cover 14 is in the cover position. When the catheter unit 100 is attached to the base unit 200, the wire cover 14 moves in the attachment-detachment direction DE from the cover position to the exposure position.

When the wire cover 14 is in the exposure position, the first to ninth held portions (Wa11 to Wa33) of the first to ninth driving wires (W11 to W33) are exposed. As a result, coupling between the bend drive portion 13 and the coupling device 21, which is described later, is allowed. When the wire cover 14 is in the exposure position, the first to ninth held portions (Wa11 to Wa33) of the first to ninth driving wires (W11 to W33) and a portion of each of the wire bodies (Wb11 to Wb33) project from the first to ninth exposure holes (14a11 to 14a33). More specifically, the first to ninth held portions (Wa11 to Wa33) project from the first to ninth exposure holes (14a11 to 14a33) in an attachment direction Da, which is described later.

As illustrated in FIG. 4B, the first to ninth driving wires (W11 to W33) are arranged along a circle (imaginary circle) having a predetermined radius and are supported by the wire guide 17, which is described later.

In the present embodiment, the catheter unit 100 includes a key shaft (key or catheter-side key) 15. The key shaft 15 extends in the attachment-detachment direction DE in the present embodiment. The wire cover 14 has a shaft hole 14b through which the key shaft 15 extends. The key shaft 15 can engage with a key receiver 22, which is described later. With the key shaft 15 engaging with the key receiver 22, a movement of the catheter unit 100 with respect to the base unit 200 in a circumferential direction of the circle (imaginary circle) along which the first to ninth driving wires (W11 to W33) are arranged is limited in a predetermined range.

In the present embodiment, the first to ninth driving wires (W11 to W33) as viewed in the attachment-detachment direction DE are disposed on the outer side of the key shaft 15 so as to surround the key shaft 15. In other words, the key shaft 15 is disposed on the inner side of the circle (imaginary circle) along which the first to ninth driving wires (W11 to W33) are arranged.

In the present embodiment, the catheter unit 100 includes the first operation portion 400. The first operation portion 400 is configured to be movable (rotatable) with respect to the proximal end cover 16 and the bend drive portion 13. The first operation portion 400 is rotatable about a rotation axis 400r. The rotation axis 400r of the first operation portion 400 extends in the attachment-detachment direction DE.

The first operation portion 400 is configured to be movable (rotatable) with respect to the base unit 200 in a state in which the catheter unit 100 is attached to the base unit 200. More specifically, the first operation portion 400 is configured to be movable (rotatable) with respect to the base housing 200f, the wire drive portion 300, and the coupling device 21, which is described later.

In addition, the catheter unit 100 includes a coupling portion cover 18, a lock member 401, a lock urging member (urging member) 402 that urges the lock member 401, and a second operation portion 404. The coupling portion cover 18 holds the lock member 401, the lock urging member 402, and the second operation portion 404.

When the catheter unit 100 is attached to the base unit 200, the lock member 401 locks the catheter unit 100 at the base unit 200 to avoid separation of the catheter unit 100 from the base unit 200. The second operation portion 404 releases the locking by the lock member 401.

Locking of the catheter unit 100 with respect to the base unit 200 and separation (detachment) of the catheter unit 100 from the base unit 200 will be described later.

### Base Unit

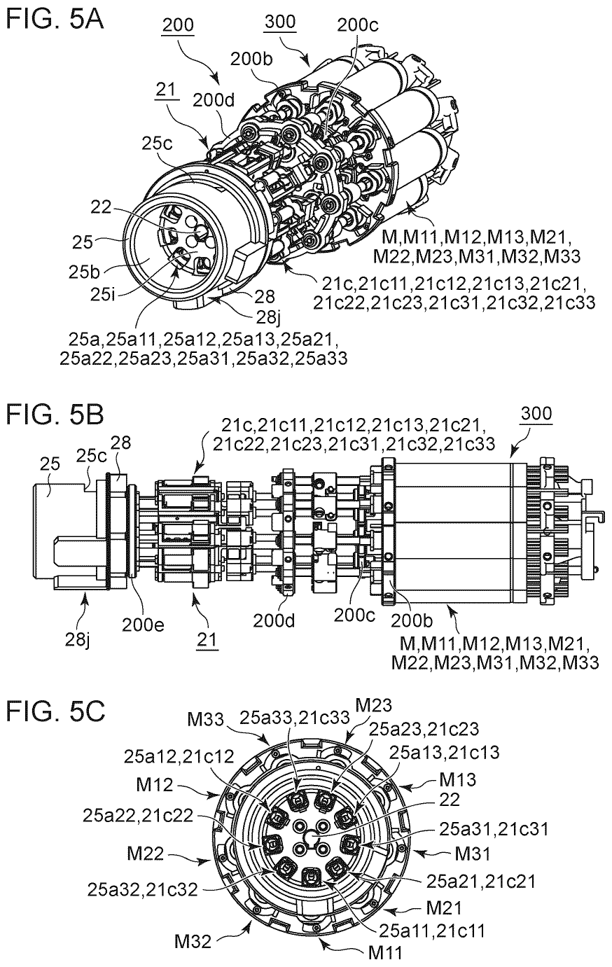

With reference to FIG. 5A to FIG. 5C, the base unit 200 and the wire drive portion 300 will be described.

FIG. 5A to FIG. 5C are explanatory views of the base unit 200 and the wire drive portion 300. FIG. 5A is a perspective view illustrating an internal structure of the base unit 200. FIG. 5B is a side view illustrating an internal structure of the base unit 200. FIG. 5C is a view of the base unit 200 as viewed in the attachment-detachment direction DE.

As described above, the medical apparatus 1 includes the base unit 200 and the wire drive portion 300. In the present embodiment, the wire drive portion 300 is housed in the base housing 200f and is included in the inside of the base unit 200. In other words, the base unit 200 includes the wire drive portion 300.

The wire drive portion 300 includes a plurality of driving sources (motors). In the present embodiment, the wire drive portion 300 includes a first driving source M11, a second driving source M12, a third driving source M13, a fourth driving source M21, a fifth driving source M22, a sixth driving source M23, a seventh driving source M31, an eighth driving source M32, and a ninth driving source M33.

Any one of the first to ninth driving sources (M11 to M33) can be referred to as the driving source M. In the present embodiment, the first to ninth driving sources (M11 to M33) have identical configurations.

The base unit 200 includes the coupling device 21. The coupling device 21 is housed in the base housing 200f. The coupling device 21 is connected to the wire drive portion 300. The coupling device 21 includes a plurality of coupling portions. In the present embodiment, the coupling device 21 includes a first coupling portion 21c11, a second coupling portion 21c12, a third coupling portion 21c13, a fourth coupling portion 21c21, a fifth coupling portion 21c22, a sixth coupling portion 21c23, a seventh coupling portion 21c31, an eighth coupling portion 21c32, and a ninth coupling portion 21c33.

Any one of the first to ninth coupling portions (21c11 to 21c33) can be referred to as the coupling portion 21c. In the present embodiment, the first to ninth coupling portions (21c11 to 21c33) have identical configurations.

The plurality of coupling portions are each connected to a corresponding one of the plurality of driving sources and driven by the corresponding one of the plurality of driving sources. Specifically, the first coupling portion 21c11 is connected to the first driving source M11 and is driven by the first driving source M11. The second coupling portion 21c12 is connected to the second driving source M12 and is driven by the second driving source M12. The third coupling portion 21c13 is connected to the third driving source M13 and is driven by the third driving source M13. The fourth coupling portion 21c21 is connected to the fourth driving source M21 and is driven by the fourth driving source M21. The fifth coupling portion 21c22 is connected to the fifth driving source M22 and is driven by the fifth driving source M22. The sixth coupling portion 21c23 is connected to the sixth driving source M23 and is driven by the sixth driving source M23. The seventh coupling portion 21c31 is connected to the seventh driving source M31 and is driven by the seventh driving source M31. The eighth coupling portion 21c32 is connected to the eighth driving source M32 and is driven by the eighth driving source M32. The ninth coupling portion 21c33 is connected to the ninth driving source M33 and is driven by the ninth driving source M33.

As described later, the bend drive portion 13 including the first to ninth driving wires (W11 to W33) is coupled to the coupling device 21. The bend drive portion 13 receives a driving force of the wire drive portion 300 through the coupling device 21 and bends the bending portion 12.

The driving wire W is coupled to the coupling portion 21c via the held portion Wa. The plurality of driving wires are each coupled to a corresponding one of the plurality of coupling portions.

Specifically, the first held portion Wa11 of the first driving wire W11 is coupled to the first coupling portion 21c11. The second held portion Wa12 of the second driving wire W12 is coupled to the second coupling portion 21c12. The third held portion Wa13 of the third driving wire W13 is coupled to the third coupling portion 21c13. The fourth held portion Wa21 of the fourth driving wire W21 is coupled to the fourth coupling portion 21c21. The fifth held portion Wa22 of the fifth driving wire W22 is coupled to the fifth coupling portion 21c22. The sixth held portion Wa23 of the sixth driving wire W23 is coupled to the sixth coupling portion 21c23. The seventh held portion Wa31 of the seventh driving wire W31 is coupled to the seventh coupling portion 21c31. The eighth held portion Wa32 of the eighth driving wire W32 is coupled to the eighth coupling portion 21c32. The ninth held portion Wa33 of the ninth driving wire W33 is coupled to the ninth coupling portion 21c33.

The base unit 200 includes a base frame 25. The base frame 25 has a plurality of insertion holes through each of which a corresponding one of the first to ninth driving wires (W11 to W33) passes. The base frame 25 has a first insertion hole 25a11, a second insertion hole 25a12, a third insertion hole 25a13, a fourth insertion hole 25a21, a fifth insertion hole 25a22, a sixth insertion hole 25a23, a seventh insertion hole 25a31, an eighth insertion hole 25a32, and a ninth insertion hole 25a33. The first to ninth insertion holes (25a11 to 25a33) correspond to the first to ninth driving wires (W11 to W33), respectively. A number after the sign 25a indicates a number of a corresponding driving wire. For example, the first driving wire W11 is inserted into the first insertion hole 25a11.

Any one of the first to ninth insertion holes (25a11 to 25a33) can be referred to as the insertion hole 25a. In the present embodiment, the first to ninth insertion holes (25a11 to 25a33) have identical shapes.

The base frame 25 has an attachment opening 25b into which the wire cover 14 is to be inserted. The first to ninth insertion holes (25a11 to 25a33) are disposed in a bottom portion 25i of the attachment opening 25b.

Further, the base unit 200 includes a motor frame 200b, a first bearing frame 200c, a second bearing frame 200d, and a third bearing frame 200e. The motor frame 200b, the first bearing frame 200c, the second bearing frame 200d, and the third bearing frame 200e are coupled to each other.

The base frame 25 has a key receiver (key hole, base-side key, or body-side key) 22 that receives the key shaft 15. With the key shaft 15 and the key receiver 22 engaging with each other, a situation in which the catheter unit 100 is attached to a wrong phase in the base unit 200 is avoided.

With the key shaft 15 and the key receiver 22 engaging with each other, a movement of the catheter unit 100 with respect to the base unit 200 in the circumferential direction of the circle (imaginary circle) along which the first to ninth driving wires (W11 to W33) are arranged is limited in a predetermined range.

As a result, the first to ninth driving wires (W11 to W33) each engage with a corresponding one of the first to ninth insertion holes (25a11 to 25a33) and with a corresponding one of the first to ninth coupling portions (21c11 to 21c33). In other words, engagement of the driving wire W with the insertion hole 25a that differs from the corresponding one of the insertion holes 25a and with the coupling portion 21c that differs from the corresponding one of the coupling portions 21c is avoided.

A user can correctly couple each of the first to ninth driving wires (W11 to W33) to the corresponding one of the first to ninth coupling portions (21c11 to 21c33) by engaging the key shaft 15 with the key receiver 22. Thus, the user can easily attach the catheter unit 100 to the base unit 200.

In the present embodiment, the key shaft 15 includes a projection that projects in a direction intersecting the attachment-detachment direction DE, and the key receiver 22 includes a recess into which the projection is to be inserted. In the circumferential direction, a position in which the projection and the recess engage with each other is a position in which the driving wire W engages with the insertion hole 25a corresponding thereto and with the coupling portion 21c corresponding thereto.

The key shaft 15 can be disposed at one of the base unit 200 and the catheter unit 100 with the key receiver 22 disposed at another one of the base unit 200 and the catheter unit 100. For example, the key shaft 15 may be disposed at the base unit 200 with the key receiver 22 disposed at the catheter unit 100.

The base unit 200 includes a joint 28 that includes a joint engagement portion 28j. The base frame 25 includes an engaged shape portion (engaged portion) 25c that engages with the lock member 401 of the catheter unit 100. With the lock member 401 engaging with the engaged shape portion 25c, separation of the catheter unit 100 from the base unit 200 is avoided.

Locking of the catheter unit 100 with respect to the base unit 200 and separation (detachment) of the catheter unit 100 from the base unit 200 will be described later.

### Coupling of Motor and Driving Wire

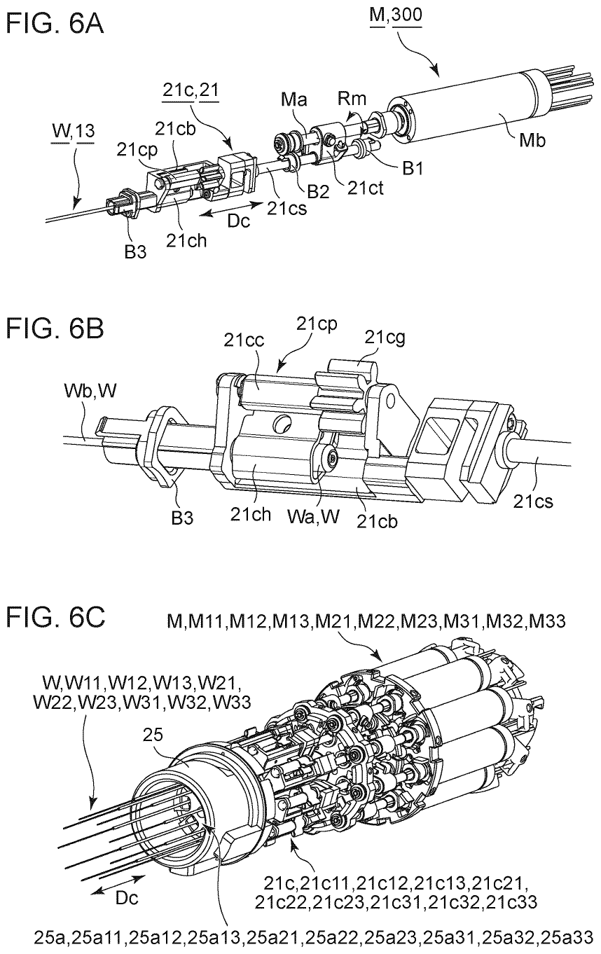

With reference to FIG. 6A to FIG. 6C, coupling of the wire drive portion 300, the coupling device 21, and the bend drive portion 13 will be described.

FIG. 6A to FIG. 6C are explanatory views of the wire drive portion 300, the coupling device 21, and the bend drive portion 13. FIG. 6A is a perspective view of the driving source M, the coupling portion 21c, and the driving wire W. FIG. 6B is an enlarged view of the coupling portion 21c and the driving wire W. FIG. 6C is a perspective view illustrating coupling of the wire drive portion 300, the coupling device 21, and the bend drive portion 13.

In the present embodiment, configurations of coupling of the first to ninth driving wires (W11 to W33) to the first to ninth coupling portions (21c11 to 21c33) corresponding thereto are identical. Configurations of connection of the first to ninth coupling portions (21c11 to 21c33) to the first to ninth driving sources (M11 to M33) corresponding thereto are also identical. Accordingly, the one driving wire W, the one coupling portion 21c, and the one driving source M are used in the following description to describe a configuration in which these are connected to each other.

As illustrated in FIG. 6A, the driving source M includes an output shaft Ma and a motor body Mb that rotates the output shaft Ma in a rotational direction Rm. A surface of the output shaft Ma has a helical groove. The output shaft Ma has a so-called screw shape. The motor body Mb is fixed to the motor frame 200b.

The coupling portion 21c includes a tractor 21ct that is connected to the output shaft Ma and a tractor supporting shaft 21cs that supports the tractor 21ct. The tractor supporting shaft 21cs is connected to a coupling base 21cb.

The coupling portion 21c includes a plate spring 21ch as a holder for holding the held portion Wa of the driving wire W. The driving wire W passes through the insertion hole 25a and engages with the coupling portion 21c. More specifically, the held portion Wa engages with the plate spring 21ch. As described later, the plate spring 21ch can enter a state (fixation state) of sandwiching and fixing the held portion Wa and a state (release state) of releasing the held portion Wa.

The coupling portion 21c includes a pressing member 21cp. The pressing member 21cp includes a gear portion 21cg that meshes with an internal gear 29, which is described later, and a cam 21cc as a pressing portion for pressing the plate spring 21ch.

As described later, the cam 21cc is movable with respect to the plate spring 21ch. The plate spring 21ch is switched between the fixation state and the release state by a movement of the cam 21cc.

The coupling portion 21c is supported by a first bearing B1, a second bearing B2, and a third bearing B3. The first bearing B1 is supported by the first bearing frame 200c of the base unit 200. The second bearing B2 is supported by the second bearing frame 200d of the base unit 200. The third bearing B3 is supported by the third bearing frame 200e of the base unit 200. Therefore, when the output shaft Ma rotates in the rotational direction Rm, a rotation of the coupling portion 21c about the output shaft Ma is restricted. Each of the first to ninth coupling portions (21c11 to 21c33) is provided with the first bearing B1, the second bearing B2, and the third bearing B3.

Since a rotation of the coupling portion 21c about the output shaft Ma is restricted, when the output shaft Ma rotates, a force in a rotation axis direction of the output shaft Ma acts on the tractor 21ct due to the helical groove of the output shaft Ma. As a result, the coupling portion 21c moves in the rotation axis direction (Dc direction) of the output shaft Ma. The driving wire W is moved by a movement of the coupling portion 21c and bends the bending portion 12.

That is, the motor shaft Ma and the tractor 21ct constitute a so-called feed screw, which converts a rotational motion transmitted from the driving source M into a linear motion by the screw. The motor shaft Ma and the tractor 21ct are sliding screws in the present embodiment but may be ball screws.

As illustrated in FIG. 6C, as a result of the catheter unit 100 being attached to the base unit 200, the first to ninth driving wires (W11 to W33) are each coupled to a corresponding one of the first to ninth coupling portions (21c11 to 21c33).

The controller 3 can control each of the first to ninth driving sources (M11 to M33) independently from each other. That is, any one of the first to ninth driving sources (M11 to M33) can independently operate or stop regardless of whether the other driving sources are in a stopped state or not. In other words, the controller 3 can control the first to ninth driving wires (W11 to W33) independently from each other. As a result, the first to third guide rings (J1 to J3) are controlled independently from each other, and the bending region 12b of the bending portion 12 can be bent in any direction.

### Attachment of Catheter Unit

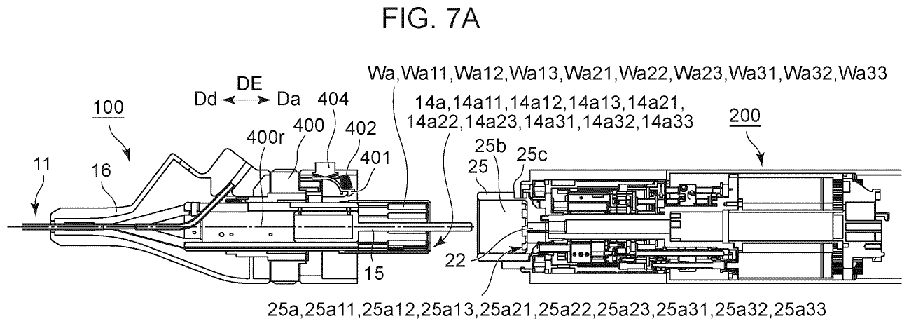

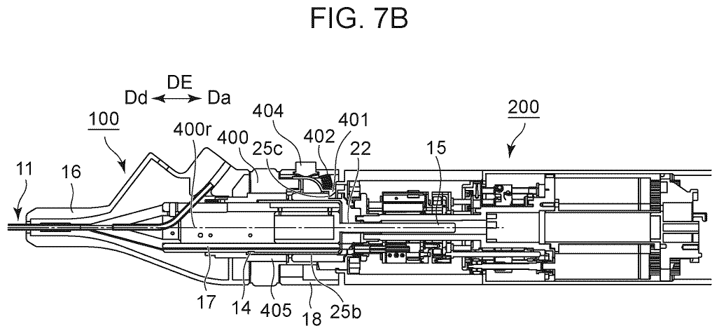

With reference to FIG. 7A and FIG. 7B, an operation of attaching the catheter unit 100 to the base unit 200 will be described.

FIG. 7A and FIG. 7B are explanatory views of attachment of the catheter unit 100. FIG. 7A is a view of the catheter unit 100 before being attached to the base unit 200. FIG. 7B is a view of the catheter unit 100 after being attached to the base unit 200.

The catheter unit 100 is detachably attached to the base unit 200. In the present embodiment, the attachment-detachment direction DE of the catheter unit 100 is identical to a direction of the rotation axis 400r of the first operation portion 400. In other words, the first operation portion 400 is rotatable about the rotation axis 400r extending in the attachment-detachment direction DE. In the attachment-detachment direction DE, a direction of attaching the catheter unit 100 to the base unit 200 is referred to as an attachment direction Da. In the attachment-detachment direction DE, a direction (a direction opposite to the attachment direction Da) of detaching the catheter unit 100 from the base unit 200 is referred to as a detachment direction Dd.

As illustrated in FIG. 7A, in a state before attachment of the catheter unit 100 to the base unit 200, the wire cover 14 is in the cover position. At this time, the wire cover 14 covers the first to ninth driving wires (W11 to W33) so that the first to ninth held portions (Wa11 to Wa33) do not project from the first to ninth exposure holes (14a11 to 14a33) of the wire cover 14.

When the key shaft 15 and the key receiver 22 engage with each other and the catheter unit 100 is moved in the attachment direction Da with respect to the base unit 200, the catheter unit 100 is attached to the base unit 200. Attaching the catheter unit 100 to the base unit 200 moves the wire cover 14 to the exposure position. In the present embodiment, the wire cover 14 moves (refer to FIG. 7B) from the cover position to the exposure position by coming into contact with the base frame 25.

More specifically, in attachment of the catheter unit 100, the wire cover 14 comes into contact with the base frame 25 and stops. By moving the catheter unit 100 in the attachment direction Da in this state, the wire cover 14 relatively moves with respect to parts other than the wire cover 14 in the catheter unit 100. As a result, the wire cover 14 moves from the cover position to the exposure position.

While the wire cover 14 moves from the cover position to the exposure position, the held portion Wa of the driving wire W projects from the exposure hole 14a of the wire cover 14 and is inserted into the insertion hole 25a. Then, the held portion Wa engages with the coupling base 21cb of the coupling portion 21c (refer to FIG. 6B).

As illustrated in FIG. 7B, with the catheter unit 100 in a stop position, the lock member 401 is in a lock position, and the lock member 401 engages with the engaged shape portion 25c. Due to these engagement relationships, a movement of the catheter unit 100 in the detachment direction Dd is avoided, and the catheter unit 100 is in a state of being attached to the base unit 200. That is, with the catheter unit 100 being linearly attached to the base unit 200 in the attachment-detachment direction DE (more specifically, the attachment direction Da), the lock member 401 engages with the engaged shape portion 25c. As a result, detachment of the catheter unit from the base unit 200 is restricted.

### Relationship of Lock Member and First Operation Portion

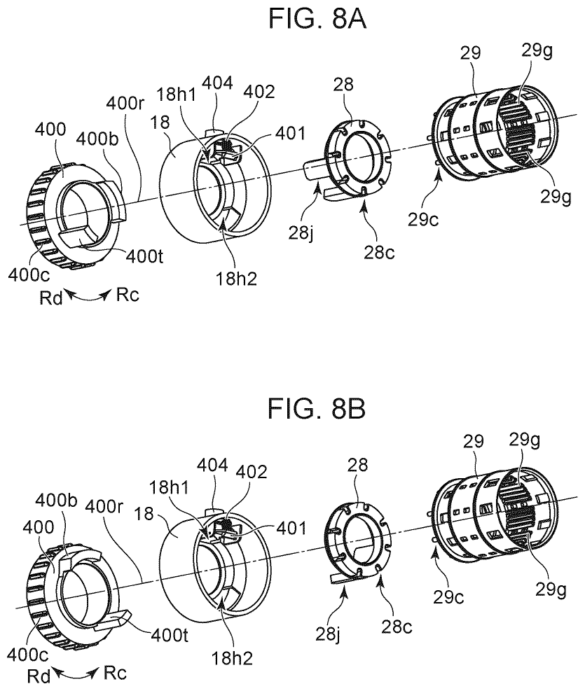

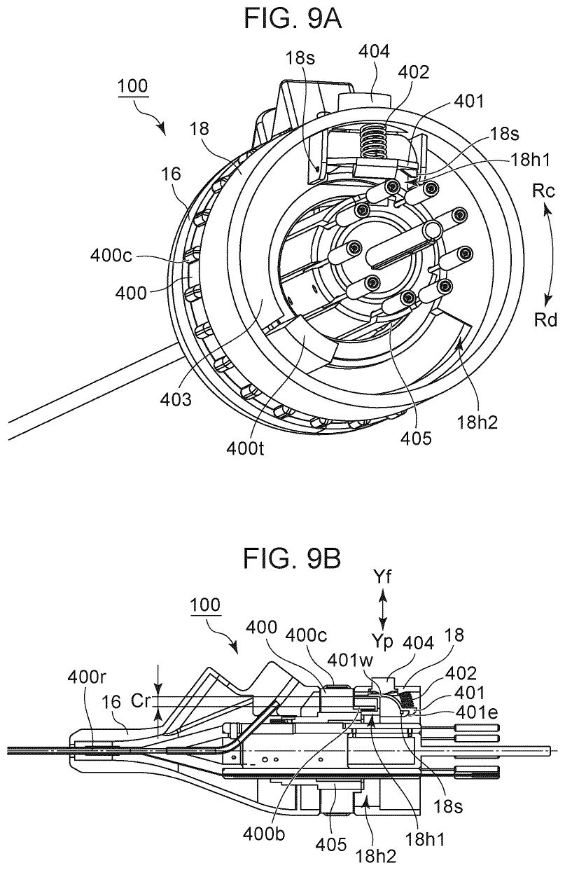

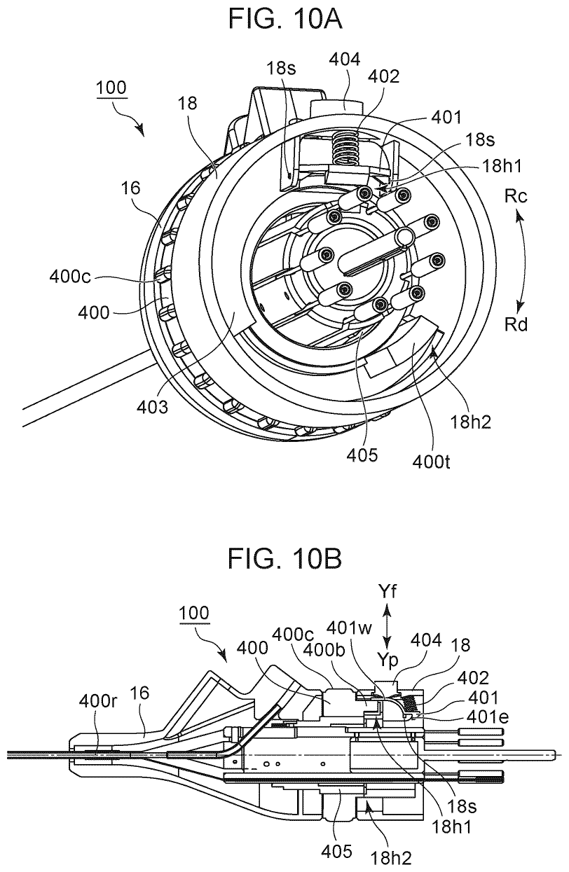

A relationship of the lock member 401 and the first operation portion 400 will be described more specifically with reference to FIG. 7A, FIG. 7B, FIG. 8A, FIG. 8B, FIG. 9A, FIG. 9B, FIG. 10A, and FIG. 10B.

As illustrated in FIG. 7B, the first operation portion 400 is supported by an operation-portion supporting frame 405 to be turnable about the rotation axis 400r.

The wire guide 17 that bends the driving wire W between the held portion Wa and the catheter 11 is disposed in the inside of the proximal end cover 16. The operation-portion supporting frame 405 and the proximal end cover 16 are fixed to the wire guide 17 so as to be integral with the wire guide 17, and the coupling portion cover 18 is fixed to the operation-portion supporting frame 405 so as to be integral with the operation-portion supporting frame 405. Therefore, the first operation portion 400 is independently turnable with respect to the operation-portion supporting frame 405, the proximal end cover 16, the wire guide 17, and the coupling portion cover 18.

FIG. 8A and FIG. 8B are exploded perspective views illustrating the first operation portion 400 and components at the periphery of the first operation portion 400.

The first operation portion 400 is in an attachment-detachment position, which is described later, in FIG. 8A, and the first operation portion 400 is in a fixation position, which is described later, in FIG. 8B.

The first operation portion 400 is movable between the attachment-detachment position and the fixation position in a state in which the catheter unit 100 is attached to the base unit 200. More specifically, the first operation portion 400 can move to the attachment-detachment position and to the fixation position by turning about the rotation axis 400r. Attachment and detachment of the catheter unit 100 with respect to the base unit 200 are performed in a state in which the first operation portion 400 is in the attachment-detachment position.

As described later, in a state in which the catheter unit 100 is attached to the base unit 200 and in which the first operation portion 400 is in the attachment-detachment position, the held portion Wa of the driving wire W is released from the coupling portion 21c. The held portion Wa of the driving wire W is fixed to the coupling portion 21c by the first operation portion 400 moving from the attachment-detachment position to the fixation position in a state in which the catheter unit 100 is attached to the base unit 200.

The first operation portion 400 is turnable in two directions of an Rc direction and an Rd direction. The first operation portion 400 includes an operation region (exposure portion or force receiver) 400c that is exposed to the outside of the catheter unit 100 and that is to be turned by a user, an operation-side engagement portion 400t, and an operation restriction portion 400b that restricts a movement of the lock member 401.

The operation region 400c has a function as a handle having a surface with irregularities and is exposed to the outside of the medical apparatus 1 in a state in which the catheter unit 100 is attached to the base unit 200. Therefore, a user can perform an operation by touching the operation region 400c, and the first operation portion 400 is rotated by the operation region 400c receiving an external force.

The coupling portion cover 18 has a communication hole 18h1 into which the operation restriction portion 400b is to be inserted and a communication hole 18h2 into which the operation-side engagement portion 400t is to be inserted. In a state of being inserted into the communication hole 18h1, the operation restriction portion 400b can be in contact with the lock member 401. The operation-side engagement portion 400t has a projecting shape and, in a state of being inserted into the communication hole 18h2, engages with the joint engagement portion 28j of the joint 28 disposed inside the base unit 200, the joint engagement portion 28j having a recessed shape. As a result, an operation force of a user turning the first operation portion 400 can be transmitted from the first operation portion 400 to the coupling device 21 of the base unit 200.

The operation-side engagement portion 400t may have a recessed shape while the joint engagement portion 28j has a projecting shape.

The base unit 200 includes the internal gear 29 as a moving gear that is in conjunction with the first operation portion 400 via the joint 28.

The joint 28 includes a plurality of transmission portions 28c, and the internal gear 29 includes a plurality of transmitted portions 29c and a tooth portion 29g. The plurality of transmission portions 28c engage with the plurality of transmitted portions 29c, and, when the joint 28 rotates, a rotation of the joint 28 is transmitted to the internal gear 29.

When the catheter unit 100 is attached to the base unit 200, the operation-side engagement portion 400t included in the first operation portion 400 engages with the joint engagement portion 28j of the joint 28. When the first operation portion 400 rotates, the rotation of the first operation portion 400 is transmitted to the joint 28, and, when the joint 28 rotates, the internal gear 29 rotates. That is, the first operation portion 400, the joint 28, and the internal gear 29 rotate in the same direction.

The tooth portion 29g of the internal gear 29 engages with the gear portion 21cg illustrated in FIG. 6B. As a result, the gear portion 21cg of the coupling device 21 is driven by an operation of the first operation portion 400. A specific operation of the coupling device 21 will be described later.

FIG. 9A, FIG. 9B, FIG. 10A, and FIG. 10B are views for describing a relationship among the first operation portion 400, the lock member 401, and the second operation portion 404. Note that illustration of the wire cover 14 is omitted for ease of understanding of description.

FIG. 9A is a perspective view of the catheter unit 100 with the first operation portion 400 in the attachment-detachment position. FIG. 9B is a sectional view of the catheter unit 100 with the first operation portion 400 in the attachment-detachment position.

FIG. 10A is a perspective view of the catheter unit 100 with the first operation portion 400 in the fixation position. FIG. 10B is a sectional view of the catheter unit 100 with the first operation portion 400 in the fixation position. In FIG. 10A, a state in which the first operation portion 400 in the state in FIG. 9A and FIG. 9B is operated in the Rc direction is illustrated.

The coupling portion cover 18 includes a lock-member supporting portion 18s that supports the lock member 401, and supports the second operation portion 404 to be movable in a direction intersecting (preferably, orthogonal to) the direction of the rotation axis 400r. In the present embodiment, the second operation portion 404 is a button that is exposed to the outside of the catheter unit 100. In a state in which the catheter unit 100 is attached to the base unit 200, the second operation portion 404 is exposed to the outside of the medical apparatus 1, and a user can operate the second operation portion 404.

The second operation portion 404 is movable in a Yf direction and a Yp direction (lock release direction). As described later, the second operation portion 404 moves in the Yp direction to thereby move the lock member 401 from the lock position in which the lock member 401 engages with the engaged shape portion 25c to an unlock position in which the lock member 401 separates from the engaged shape portion 25c.

The second operation portion 404 is positioned away from the first operation portion 400, and the second operation portion 404 and the first operation portion 400 are movable independently from each other. That is, in a state in which one of the second operation portion 404 and the first operation portion 400 stops, the other one is movable.

The lock member 401 is swingable (turnable) between the unlock position and the lock position about the lock-member supporting portion 18s. The lock member 401 is urged in a direction from the unlock position toward the lock position by the lock urging member 402.

The lock member 401 includes a lock engagement portion 401e and an operation receiver 401w. The second operation portion 404 is in contact with the operation receiver 401w of the lock member 401 and is urged by the lock urging member 402 via the lock member 401 in the Yf direction of being exposed to the outside of the apparatus.

In the state illustrated in FIG. 9B, the first operation portion 400 is in the attachment-detachment position, and a gap Cr is formed between the lock member 401 and the operation restriction portion 400b. Therefore, the lock member 401 can be turned against an urging force of the lock urging member 402 by the second operation portion 404 being operated in the Yp direction.

In contrast to FIG. 9A and FIG. 9B, FIG. 10A and FIG. 10B illustrate a state in which the operation restriction portion 400b and the operation-side engagement portion 400t are turned about the rotation axis 400r by an operation of the first operation portion 400 and in which the first operation portion 400 is in the fixation position. As described later, the held portion Wa is fixed to the coupling portion 21c by the operation-side engagement portion 400t moving in a state in which the catheter unit 100 is attached to the base unit 200.

As illustrated in FIG. 10B, the gap Cr formed between the lock member 401 and the operation restriction portion 400b is eliminated by the operation restriction portion 400b. Specifically, the operation restriction portion 400b moves to a position in which the operation restriction portion 400b restricts the operation receiver 401w, and a turn of the lock member 401 is restricted. With a movement of the lock member 401 being restricted, a movement of the second operation portion 404 in the Yp direction is also restricted, and an operation of the second operation portion 404 is disabled.

Attachment of Catheter Unit and Operation of Lock Member

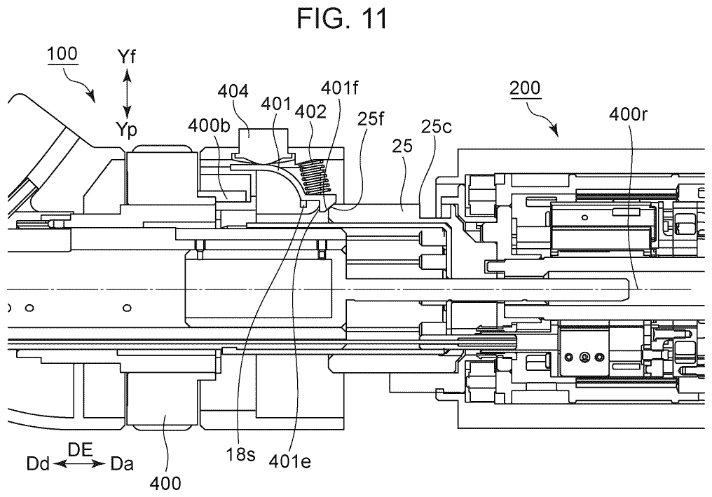

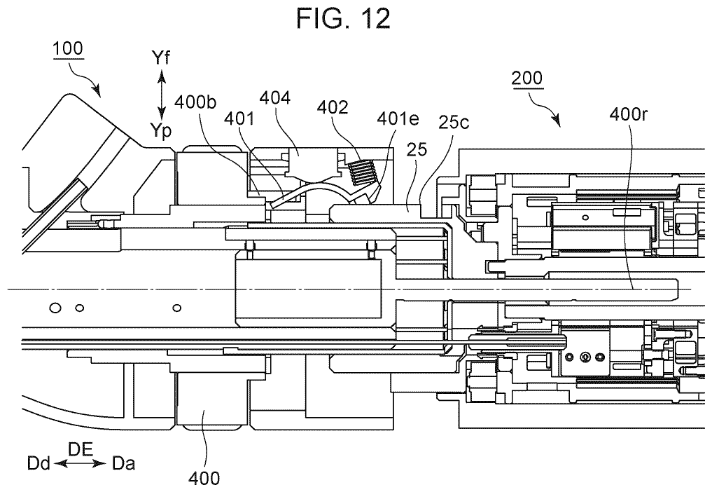

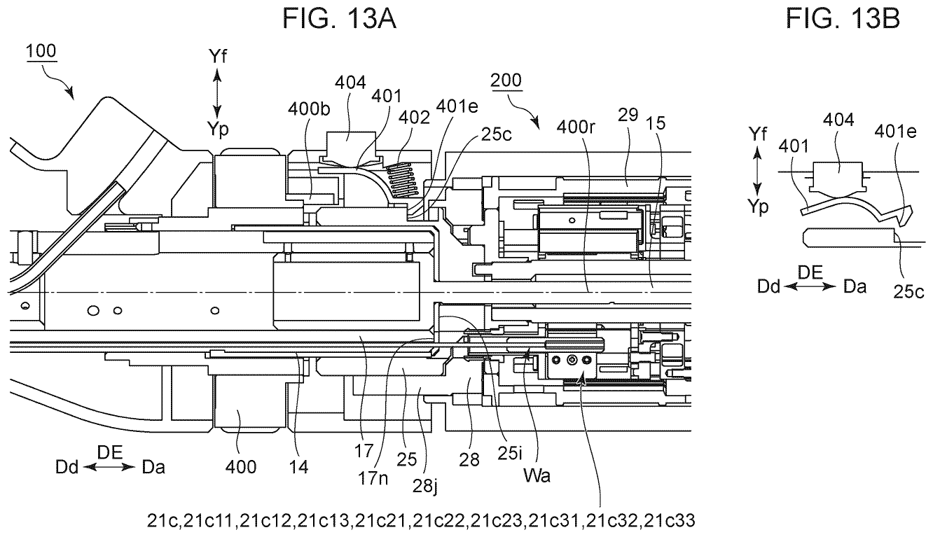

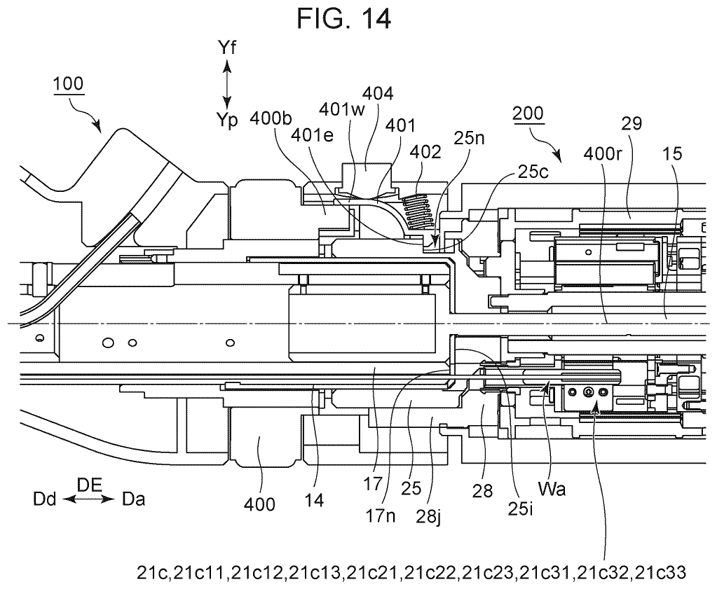

Next, with reference to FIG. 11, FIG. 12, FIG. 13A, FIG. 13B, and FIG. 14, an operation of the lock member 401 in the process of attachment of the catheter unit 100 to the base unit 200 will be described. FIG. 11 to FIG. 14 are sectional views of the catheter unit 100 and the base unit 200.

In FIG. 11, a state in which the lock member 401 is in contact with the base frame 25 in the process of insertion of the catheter unit 100 is illustrated. The first operation portion 400 at this time is in the same state as the state illustrated in FIG. 9A and FIG. 9B. Thus, the first operation portion 400 is in the attachment-detachment position, and a turn of the lock member 401 is not restricted.

The catheter unit 100 is attached to the base unit 200 by moving in the attachment direction Da. At this time, due to contact of a contacted surface 401f of the lock member 401 with a leading-end-shape portion 25f of the base frame 25, a force in the Yf direction acts on the contacted surface 401f of the lock member 401. Therefore, the lock member 401 turns about the lock-member supporting portion 18s illustrated in FIG. 9A or FIG. 10A against urging by the lock urging member 402. That is, in attaching of the catheter unit 100 to the base unit 200, the lock member 401 is moved from the lock position toward the unlock position against urging by the lock urging member 402.

The contacted surface 401f is moved to a position in which the contacted surface 401f is not in contact with the leading-end-shape portion 25f by a turn of the lock member 401, thereby enabling attachment of the catheter unit 100 to the base unit 200 to be continued.

In FIG. 12, a state in which insertion of the catheter unit 100 has progressed from the state illustrated in FIG. 11. The orientation of the lock member 401 is restricted by being in contact with the outer periphery of the base frame 25 while receiving an urging force of the lock urging member 402. The lock member 401 at this time is in the same position as the unlock position.

In the state in FIG. 12, if the first operation portion 400 is attempted to be operated, a rotation of the first operation portion 400 is obstructed due to presence of the lock member 401 on a track of the operation restriction portion 400b. That is, the first operation portion 400 is not operable in the state in FIG. 12.

That is, when the lock member 401 is in the unlock position, the lock member 401 restricts a movement (rotation) of the first operation portion 400. Consequently, a rotation of the first operation portion 400 after a portion (lock member 401) of the catheter unit 100 coming into contact with the base unit 200 and a rotation of the first operation portion 400 before the catheter unit 100 reaching an attachment completion position are restricted.

In FIG. 13A and FIG. 13B, a state in which the catheter unit 100 has reached the attachment completion position with respect to the base unit 200 is illustrated. In FIG. 13A, the first operation portion 400 is in the attachment-detachment position, and the lock member 401 is in the lock position. In FIG. 13B, a state in which, with the catheter unit 100 attached to the base unit 200, the second operation portion 404 is moved in the Yp direction and the lock member 401 is in the unlock position is illustrated.

The attachment completion position in the present embodiment is specifically a position in which a proximal end 17n of the wire guide 17 comes into contact via the wire cover 14 with the bottom portion 25i of the attachment opening 25b of the base frame 25 illustrated in FIG. 5A.

In a state in which the catheter unit 100 is attached to the base unit 200 and in which the first operation portion 400 is in the attachment-detachment position, the lock member 401 is movable to the lock position in which the lock member 401 restricts detachment of the catheter unit 100 from the base unit 200 and to the unlock position. Detachment of the catheter unit 100 from the base unit 200 is allowed in a state in which the catheter unit 100 is attached to the base unit 200, in which the first operation portion 400 is in the attachment-detachment position, and in which the lock member 401 is in the unlock position.

As illustrated in FIG. 13A, when the catheter unit 100 reaches the attachment completion position, the lock member 401 is urged by the lock urging member 402 such that the lock engagement portion 401e engages with the engaged shape portion 25c. In this state, the lock member 401 is in the lock position, and a movement of the catheter unit 100 with respect to the base unit 200 in the direction of the rotation axis 400r is restricted. That is, the catheter unit 100 is not movable in the detachment direction Dd with respect to the base unit 200, and it is not possible to detach the catheter unit 100 from the base unit 200.

As described above, with the catheter unit 100 being linearly attached to the base unit 200 in the attachment direction Da, the catheter unit 100 is in a state in which detachment of the catheter unit 100 from the base unit 200 is restricted. As described above, the detachment direction Dd and the attachment direction Da extend in the direction of the rotation axis 400r.

In the state illustrated in FIG. 13A, the held portion Wa of the driving wire W reaches the position illustrated in FIG. 6B with respect to the coupling portion 21c of the coupling device 21. However, the first operation portion 400 is not moved to the fixation position, and the held portion Wa is in a state of not being fixed to the coupling portion 21c. That is, a force of fixing the held portion Wa to the coupling portion 21c is not required for attachment of the catheter unit 100 to the base unit 200. Therefore, a force of attaching the catheter unit 100 to the base unit 200 is suppressed to be small.

Meanwhile, moving the first operation portion 400 from the attachment-detachment position to the fixation position by operating the first operation portion 400 in the state in FIG. 13A fixes the held portion Wa to the coupling portion 21c. As described above, a movement of the first operation portion 400 is limited in the state in FIG. 12. Thus, fixation of the held portion Wa to the coupling portion 21c before the catheter unit 100 reaches the attachment completion position is limited.

In FIG. 14, a state in which the first operation portion 400 is operated after the catheter unit 100 is attached to the base unit 200 is illustrated. In FIG. 14, the first operation portion 400 is in the fixation position, and the lock member 401 is in the lock position. The catheter unit 100 at this time is in the same state as the state in FIG. 10A and FIG. 10B. That is, a movement of the lock member 401 is limited in the state.

As described later, since the driving wire W is coupled to the driving source M, the first operation portion 400 is moved to the fixation position, and the held portion Wa of the driving wire W is fixed to the coupling portion 21c. As described with reference to FIG. 10A and FIG. 10B, in a state in which the first operation portion 400 is in the fixation position, a movement of the lock member 401 from the lock position to the unlock position is restricted by the operation restriction portion 400b. Therefore, in a state in which the catheter unit 100 is attached to the base unit 200 and in which the first operation portion 400 is in the fixation position, a movement of the lock member 401 from the lock position to the unlock position is restricted. At the same time, due to the movement of the lock member 401 being restricted, an operation (a movement in the Yp direction) of the second operation portion 404 that moves the lock member 401 from the lock position to the unlock position is also restricted.

In the present embodiment, an operation in which the catheter unit 100 is attached to the base unit 200 while the base unit 200 is in a horizontal state (a state in which the direction of the rotation axis 400r is the horizontal direction) has been described. However, usage environment of the medical apparatus 1 includes a situation in which attachment of the catheter unit 100 is performed in a state in which the base unit 200 is inclined. In the present embodiment, the support base 2 of the medical system 1A can support the medical apparatus 1 to be inclined such that the catheter unit 100 is positioned below the base unit 200 in the vertical direction. In this case, the catheter unit 100 is detached from the base unit 200 by being moved downward in the vertical direction.

However, as illustrated in FIG. 11 to FIG. 13, with the catheter unit 100 being attached to the base unit 200, detachment of the catheter unit 100 from the base unit 200 is limited by the lock member 401. Accordingly, an operation of the first operation portion 400 is not required for locking the catheter unit 100 to the base unit 200. In addition, the catheter unit 100 does not come off from the base unit 200 during an operation of the first operation portion 400.

As illustrated in FIG. 13A, in a state in which the catheter unit 100 is simply attached to the base unit 200, the operation restriction portion 400b is in a position in which a turn of the lock member 401 is not obstructed. Thus, a user can turn the lock member 401 from the lock position to the unlock position (refer to FIG. 13B) by pressing the second operation portion 404 in the Yp direction. Meanwhile, the held portion Wa is in a state of not being fixed to the coupling portion 21c. Therefore, a user can detach the catheter unit 100 by, in a state in which the second operation portion 404 is pressed in the Yp direction, moving the catheter unit 100 with respect to the base unit 200 in the detachment direction Dd.

Further, as illustrated also in FIG. 12, the first operation portion 400 in the present embodiment is provided to be turnable about the rotation axis 400r, and the second operation portion 404 is provided independently from the first operation portion 400.

As described above, a position in which the lock member 401 does not allow detachment of the catheter unit 100 is referred to as the lock position, and a position in which the lock member 401 allows detachment of the catheter unit 100 is referred to as the unlock position. The lock member 401 is moved from the lock position to the unlock position by an operation of the second operation portion 404.

### Fixation and Release of Bend Drive Portion

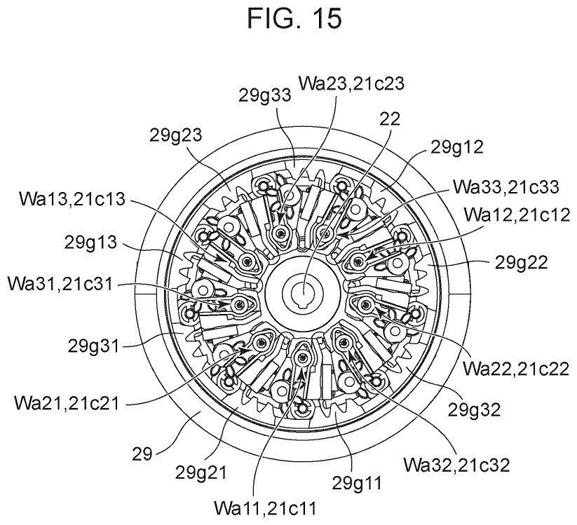

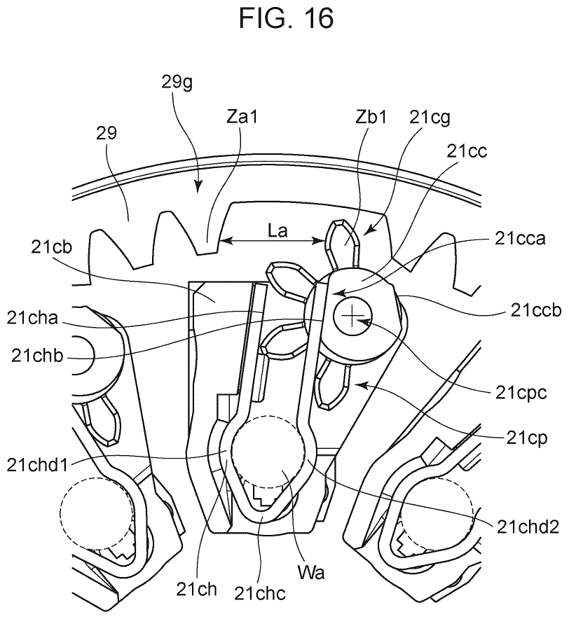

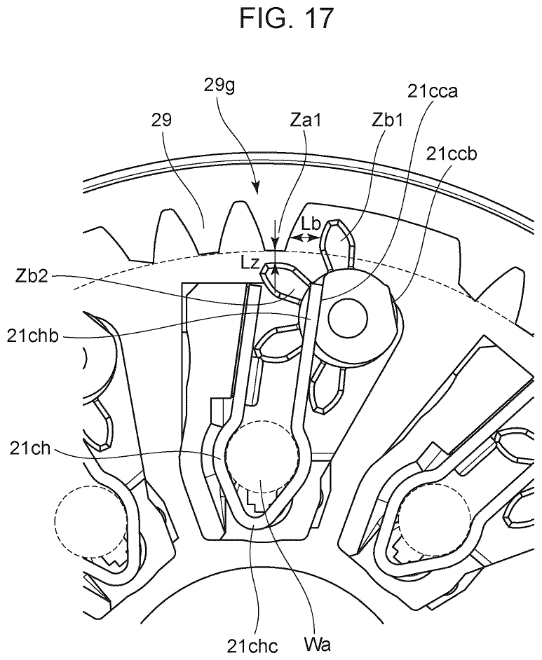

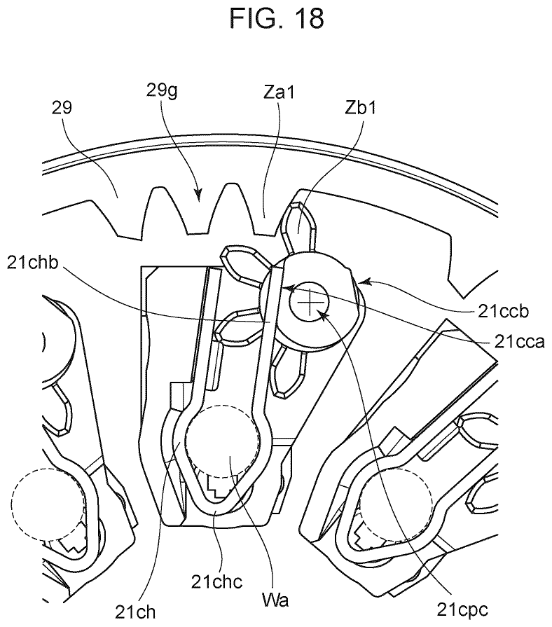

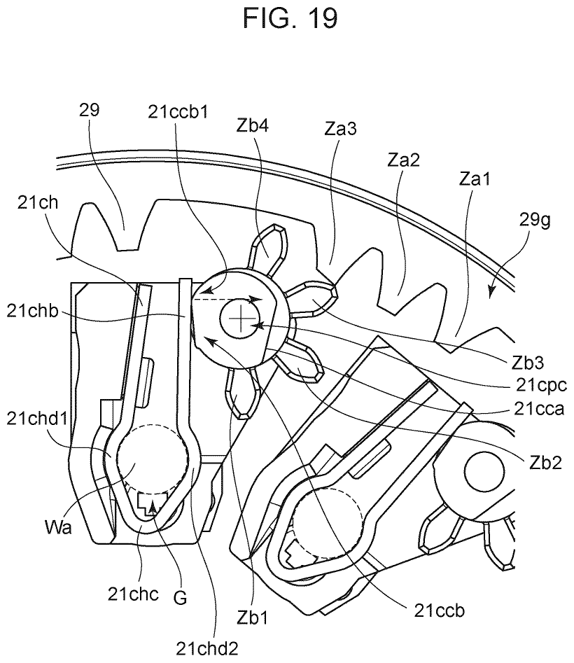

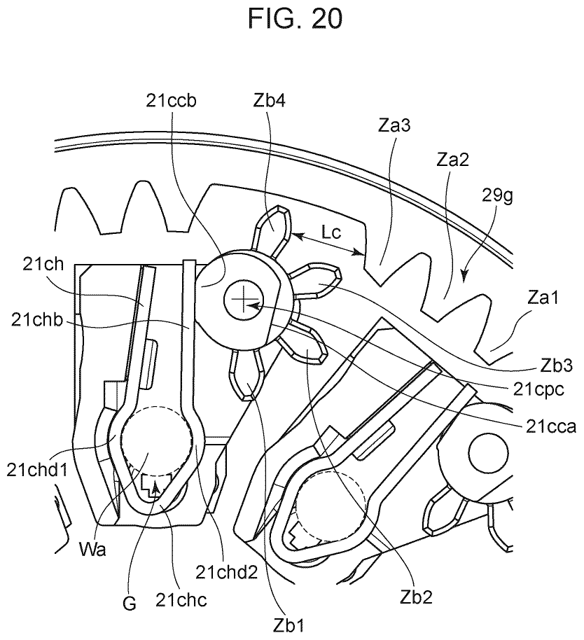

With reference to FIG. 15, FIG. 16, FIG. 17, FIG. 18, FIG. 19, and FIG. 20A, configuration for fixing the bend drive portion 13 to the coupling device 21 and a configuration for releasing the bend drive portion 13 from the coupling device 21 will be described.

FIG. 15 is a sectional view of the base unit 200 and is a sectional view in which the base unit 200 is cut at a portion of the coupling portion 21c in a direction orthogonal to the rotation axis 400r.

FIG. 16, FIG. 17, FIG. 18, FIG. 19, and FIG. 20 are views for describing fixation of the driving wire W by the coupling portion 21c.

As described with reference to FIG. 8A and FIG. 8B, the coupling portion 21c of the coupling device 21 is driven via the joint 28 and the internal gear 29 by an operation of the first operation portion 400.

The internal gear 29 includes a plurality of tooth portions for switching between a state in which each of the first to ninth coupling portions (21c11 to 21c33) fixes a corresponding one of the first to ninth driving wires (W11 to W33) and a state in which each of the first to ninth coupling portions (21c11 to 21c33) releases the corresponding one of the first to ninth driving wires (W11 to W33). The plurality of tooth portions (action portions or switching gear portions) of the internal gear 29 each engage with the gear portion 21cg of the pressing member 21cp of a corresponding one of the first to ninth coupling portions (21c11 to 21c33).

Specifically, as illustrated in FIG. 15, the internal gear 29 in the present embodiment includes a first tooth portion 29g11, a second tooth portion 29g12, a third tooth portion 29g13, a fourth tooth portion 29g21, a fifth tooth portion 29g22, a sixth tooth portion 29g23, a seventh tooth portion 29g31, an eighth tooth portion 29g32, and a ninth tooth portion 29g33. The first to ninth tooth portions (29g11 to 29g33) are formed with a gap therebetween.

The first tooth portion 29g11 meshes with the gear portion 21cg of the first coupling portion 21c11. The second tooth portion 29g12 meshes with the gear portion 21cg of the second coupling portion 21c12. The third tooth portion 29g13 meshes with the gear portion 21cg of the third coupling portion 21c13. The fourth tooth portion 29g21 meshes with the gear portion 21cg of the fourth coupling portion 21c21. The fifth tooth portion 29g22 meshes with the gear portion 21cg of the fifth coupling portion 21c22. The sixth tooth portion 29g23 meshes with the gear portion 21cg of the sixth coupling portion 21c23. The seventh tooth portion 29g31 meshes with the gear portion 21cg of the seventh coupling portion 21c31. The eighth tooth portion 29g32 meshes with the gear portion 21cg of the eighth coupling portion 21c32. The ninth tooth portion 29g33 meshes with the gear portion 21cg of the ninth coupling portion 21c33.

Any one of the first to ninth tooth portions (29g11 to 29g33) can be referred to as the tooth portion 29g. In the present embodiment, the first to ninth tooth portions (29g11 to 29g33) have identical configurations.

In the present embodiment, configurations of coupling of the first to ninth driving wires (W11 to W33) to the first to ninth coupling portions (21c11 to 21c33) corresponding thereto is identical. Configurations of connection of the first to ninth coupling portions (21c11 to 21c33) to the first to ninth tooth portions (29g11 to 29g33) corresponding there to is also identical. Accordingly, the one driving wire W, the one coupling portion 21c, and the one tooth portion 29g are used in the following description to describe a configuration in which these are connected to each other.

In each of the first to ninth coupling portions (21c11 to 21c33), moving the gear portion 21cg by the internal gear 29 rotates the pressing member 21cp and causes the cam 21cc to move to a pressing position or to a retracted position in which the cam 21cc is retracted from the pressing position.

Rotating the first operation portion 400 rotates the internal gear 29. The rotation of the internal gear 29 causes each of the first to ninth coupling portions (21c11 to 21c33) to operate. That is, an operation of rotating the one first operation portion 400 can cause the first to ninth coupling portions (21c11 to 21c33) to operate.

In a state in which the catheter unit 100 is attached to the base unit 200, the first operation portion 400 is movable to the fixation position (coupling position) and to the attachment-detachment position. In addition, as described later, in the state in which the catheter unit 100 is attached to the base unit 200, the first operation portion 400 is also movable to a release position. In a circumferential direction of the first operation portion 400, the release position is between the fixation position and the attachment-detachment position. The catheter unit 100 is attached to the base unit 200 in a state in which the first operation portion 400 is in the attachment-detachment position.

In a state in which the catheter unit 100 is attached to the base unit 200, the driving wire W is in a state of being released (disconnected) from the coupling portion 21c. This state is referred to as the release state (disconnection state) of the coupling portion 21c. A state in which the driving wire W is fixed (coupled) to the coupling portion 21c is referred to as the fixation state (coupling state) of the coupling portion 21c.

With reference to FIG. 16, FIG. 17, FIG. 18, FIG. 19, and FIG. 20, an operation of fixing the driving wire W to the coupling portion 21c will be described.

In a state in which the first operation portion 400 is in the attachment-detachment position, the catheter unit 100 is attachable to the base unit 200. Therefore, the first operation portion 400 is in the attachment-detachment position in a state after attachment of the catheter unit 100 to the base unit 200 and before an operation of the first operation portion 400. In this state, the catheter unit 100 is detachable from the base unit 200 when locking by the lock member 401 is released.

In FIG. 16, the internal gear 29 and the coupling portion 21c in a state in which the first operation portion 400 is in the attachment-detachment position are illustrated.

The plate spring 21ch of the coupling portion 21c includes a fixed portion 21cha that is fixed to the coupling base 21cb, and a pressed portion 21chb that comes into contact with the cam 21cc of the pressing member 21cp. The plate spring 21ch includes a first portion 21chd1 and a second portion 21chd2. When the catheter unit 100 is attached to the base unit 200, the held portion Wa is inserted into a space between the first portion 21chd1 and the second portion chd2.

The cam 21cc has a holding surface 21cca and a pressing surface 21ccb. In the rotation radius direction of the pressing member 21cp, the holding surface 21cca is positioned closer than the pressing surface 21ccb to a rotation center 21cpc of the pressing member 21cp.

As illustrated in FIG. 16, in a state in which the first operation portion 400 is in the attachment-detachment position, the plate spring 21ch is held in a position in which the pressed portion 21chb is in contact with the holding surface 21cca. In addition, a tooth Za1 of the internal gear 29 and a tooth Zb1 of the gear portion 21cg are stopped in a state in which a clearance La is formed therebetween.

In the rotation direction of the first operation portion 400, a direction in which the first operation portion 400 moves from the attachment-detachment position to the release position and to the fixation position is referred to as a coupling direction (fixation direction), and a direction in which the first operation portion 400 moves from the fixation position to the release position and to the attachment-detachment position is referred to as a release direction (disconnection direction). The first operation portion 400 moves from the release position to the attachment-detachment position by rotating in the release direction. The first operation portion 400 moves from the release position to the fixation position by rotating in the coupling direction.

In a state in which the catheter unit 100 is attached to the base unit 200 and in which the first operation portion 400 is in the attachment-detachment position, the coupling portion 21c is in the release state, and the driving wire W is released from the coupling portion 21c. The state in which the driving wire W is released from the coupling portion 21c is a state in which transmission of the driving force of the driving source M to the driving wire W is blocked and is a state in which a movement of the driving wire W in the attachment-detachment direction DE with respect to the coupling portion 21c is allowed.

When the coupling portion 21c is in the release state, the cam 21cc is in the retracted position in which the cam 21cc is retracted from the pressing position, which is described later. At this time, the held portion Wa is released by the plate spring 21ch. A force of tightening the held portion Wa by the first portion 21chd1 and the second portion 21chd2 when the coupling portion 21c is in the release state is smaller than a force of tightening the held portion Wa by the first portion 21chd1 and the second portion 21chd2 when the coupling portion 21c is in a lock state.

When the first operation portion 400 is in the attachment-detachment position, the coupling portion 21c is in the release state while it is possible to release locking by the lock member 401 by operating the second operation portion 404. That is, when the first operation portion 400 is in the attachment-detachment position, it is possible to release locking by the lock member 401 by moving the lock member 401 from the lock position to the unlock position.

The catheter unit 100 is movable in the detachment direction Dd with respect to the base unit 200. As a result, it is possible to extract the held portion Wa from the space between the first portion 21chd1 and the second portion 21chd2 by moving the driving wire W in the detachment direction Dd with respect to the coupling portion 21c.

Preferably, when the coupling portion 21c is in the release state, the force of tightening the held portion Wa by the first portion 21chd1 and the second portion 21chd2 is not generated (the magnitude of the force is zero). Preferably, when the coupling portion 21c is in the release state, a gap is formed between the held portion Wa and at least one of the first portion 21chd1 and the second portion 21chd2.

In FIG. 17, a state of the internal gear 29 and the coupling portion 21c when the first operation portion 400 is rotated in the coupling direction from the attachment-detachment position is illustrated. In FIG. 17, a state of the internal gear 29 and the coupling portion 21c with the first operation portion 400 in the release position is illustrated.

When the first operation portion 400 is rotated in the coupling direction in a state (FIG. 16) in which the first operation portion 400 is in the attachment-detachment position, the internal gear 29 rotates clockwise. In addition, the first operation portion 400 moves to the release position.

Due to the key shaft 15 and the key receiver 22 engaging with each other, a rotation of the entirety (except for the first operation portion 400) of the catheter unit 100 with respect to the base unit 200 is restricted even when the first operation portion 400 is rotated. That is, in a state in which the entirety (except for the first operation portion 400) of the catheter unit 100 and the base unit 200 are stopped, the first operation portion 400 is rotatable with respect to the entirety of the catheter unit 100 and the base unit 200.

As a result of the internal gear 29 rotating clockwise, the clearance between the tooth Za1 of the internal gear 29 and the tooth Zb1 of the gear portion 21cg decreases from the clearance La to a clearance Lb.

A tooth Zb2 of the gear portion 21cg is positioned with a clearance Lz between the tooth Zb2 and a tooth tip circle (dotted line) of the tooth portion 29g of the internal gear 29. Therefore, the internal gear 29 is rotatable without interfering with the tooth Zb2. Meanwhile, the coupling portion 21c is maintained in the same state (release state) as the state illustrated in FIG. 10A and FIG. 10B.

When the first operation portion 400 in the state illustrated in FIG. 17 is further rotated in the coupling direction, the internal gear 29 further rotates clockwise. A state of the internal gear 29 and the coupling portion 21c at this time is illustrated in FIG. 18.

In FIG. 18, a state of the internal gear 29 and the coupling portion 21c when the first operation portion 400 is rotated from the release position in the coupling direction is illustrated.

When the first operation portion 400 is rotated from the release position in the coupling direction, the tooth Za1 of the internal gear 29 comes into contact with the tooth Zb1 of the gear portion 21cg. Meanwhile, the coupling portion 21c is in the same state as the state illustrated in FIG. 16 and FIG. 17 and is maintained in the release state.

In FIG. 19, a state in which the pressing member 21cp is rotated by a rotation of the first operation portion 400 in the coupling direction is illustrated.

As illustrated in FIG. 19, when the first operation portion 400 is further rotated from the state in FIG. 18 in the coupling direction, the internal gear 29 further rotates clockwise.

As a result of the internal gear 29 moving from the position in FIG. 18 to the position in FIG. 19, the internal gear 29 rotates the gear portion 21cg clockwise. When the gear portion 21cg is rotated, the holding surface 21cca separates from the pressed portion 21chb, and the pressing surface 21ccb approaches the pressed portion 21chb. Then, the first portion 21chd1 and the second portion 21chd2 start to sandwich the held portion Wa.

Then, a tooth Za3 of the internal gear 29 moves to a position in which, while the pressed portion 21chb is pressed by a corner portion 21ccb1 disposed at an end portion of the pressing surface 21ccb, the tooth Za3 separates from a tooth Zb3 of the gear portion 21cg. At this time, the held portion Wa is sandwiched between the first portion 21chd1 and the second portion 21chd2.

When the tooth Za3 of the internal gear 29 separates from the tooth Zb3 of the gear portion 21cg, transmission of the driving force from the internal gear 29 to the gear portion 21cg stops. At this time, the cam 21cc is in a state in which the corner portion 21ccb1 receives a reaction force from the plate spring 21ch.

In the rotation radius direction of the pressing member 21cp, the reaction force of the plate spring 21ch acting on the corner portion 21ccb1 acts at a position away from the rotation center 21cpc of the pressing member 21cp, and the pressing member 21cp rotates clockwise. At this time, the pressing member 21cp rotates in the same direction as a direction in which the pressing member 21cp is rotated by the internal gear 29 that rotates clockwise.

In FIG. 20, a state of the internal gear 29 and the coupling portion 21c with the first operation portion 400 in the fixation position is illustrated.

As illustrated in FIG. 20, the pressing member 21cp in the state illustrated in FIG. 19 further rotates by receiving the reaction force of the plate spring 21ch.

As illustrated in FIG. 20, the pressing member 21cp stops in a state in which the pressing surface 21ccb of the cam 21cc is in surface contact with the pressed portion 21chb of the plate spring 21ch. That is, the pressing surface 21ccb and a surface of the pressed portion 21chb are arranged on an identical plane.

At this time, the coupling portion 21c is in the fixation state. When the coupling portion 21c is in the fixation state, the cam 21cc of the pressing member 21cp is in the pressing position, and the pressing surface 21ccb presses the pressed portion 21chb.

When the coupling portion 21c is in the fixation state, the held portion Wa is sandwiched between the first portion 21chd1 and the second portion 21chd2. That is, the plate spring 21ch is pressed by the cam 21cc, and the held portion Wa is tightened by the plate spring 21ch. As a result, the held portion Wa is fixed by the plate spring 21ch. The state in which the held portion Wa is fixed by the plate spring 21ch is a state in which the driving force of the driving source M can be transmitted to the driving wire W.

In the present embodiment, the first portion 21chd1 and the second portion 21chd2 of the plate spring 21ch press the held portion Wa at positions away from each other. Further, a bent portion 21chc connecting the first portion 21chd1 and the second portion 21chd2 to each other is disposed between the first portion 21chd1 and the second portion 21chd2. The bent portion 21chc is disposed to be separated from the held portion Wa with a gap G therebetween. Consequently, the held portion Wa can be stably fixed by the first portion 21chd1 and the second portion 21chd2.

As a material of the plate spring 21ch, a resin or a metal is usable while it is preferable to use a metal.

When the coupling portion 21c is in the fixation state, extraction of the held portion Wa from the space between the first portion 21chd1 and the second portion 21chd2 is limited.

The tooth Za3 of the internal gear 29 and a tooth Zb4 of the gear portion 21cg are stopped at positions at which a clearance Lc is formed therebetween.

To release the driving wire W from the coupling portion 21c, the first operation portion 400 in the fixation position is rotated in the release direction. At this time, the internal gear 29 rotates counterclockwise from the state illustrated in FIG. 20. When the internal gear 29 rotates counterclockwise, the tooth Za3 of the internal gear 29 comes into contact with the tooth Zb4 of the gear portion 21cg, and the pressing member 21cp is rotated counterclockwise.

With the internal gear 29 being further rotated counterclockwise, the driving wire W is released from the coupling portion 21c. Operations of the internal gear 29 and the pressing member 21cp at this time are reverse operations of the above-described operations. That is, the driving wire W is released from the coupling portion 21c by reverse operations of the above-described operations for fixing the driving wire W by the coupling portion 21c.

The aforementioned operations are performed in each of the first to ninth coupling portions (21c11 to 21c33). That is, in the process of a movement of the first operation portion 400 from the attachment-detachment position to the fixation position, the first to ninth coupling portions (21c11 to 21c33) in the release state are caused to be in the fixation state by the movement (rotation) of the first operation portion 400. In the process of a movement of the first operation portion 400 from the fixation position to the attachment-detachment position, the first to ninth coupling portions (21c11 to 21c33) are changed from the fixation state to the release state by the movement (rotation) of the first operation portion 400. That is, a user can switch the plurality of coupling portions between the release state and the fixation state by operating the one first operation portion 400.

That is, it is not necessary to provide each of the plurality of coupling portions with an operation portion for switching between the release state and the fixation state and is not necessary for a user to operate the operation portions. Therefore, a user can easily attach and detach the catheter unit 100 to and from the base unit 200. Further, the medical apparatus 1 can be simplified.

A state in which the first to ninth driving wires (W11 to W33) are each fixed by a corresponding one of the first to ninth coupling portions (21c11 to 21c33) is referred to as a first state. A state in which each of the first to ninth driving wires (W11 to W33) is released from the corresponding one of the first to ninth coupling portions (21c11 to 21c33) is referred to as a second state.

The first state and the second state are switched in conjunction with a movement of the first operation portion 400. That is, the first state and the second state are switched in conjunction with a movement of the first operation portion 400 between the attachment-detachment position and the fixation position.

The internal gear 29 is configured to be in conjunction with the first operation portion 400. In the present embodiment, the joint 28 functions as a transmission member for causing the internal gear 29 to be in conjunction with the first operation portion 400. The internal gear 29 and the joint 28 have a function as a conjunction portion that is in conjunction with the first operation portion 400 such that the first state and the second state are switched in conjunction with a movement of the first operation portion 400.

Specifically, in a state in which the catheter unit 100 is attached to the base unit 200, the internal gear 29 and the joint 28 move a portion (the pressed portion 21chb) of the plate spring 21ch with respect to the held portion Wa in conjunction with a movement of the first operation portion 400. The movement of the pressed portion 21chb switches the coupling portion 21c between the lock state and the release state.

The internal gear 29 may be configured to be directly moved from the first operation portion 400. In this case, the internal gear 29 has a function as the conjunction portion.

### Avoidance of Detachment of Catheter Unit

In a state in which the catheter unit 100 is attached to the base unit 200 and in which the driving wire W is fixed to the coupling portion 21c, detachment of the catheter unit 100 from the base unit 200 is avoided. As described above, the state in which the catheter unit 100 is attached to the base unit 200 and in which the first operation portion 400 is in the fixation position is illustrated in FIG. 14, and the driving wire W is fixed to the coupling portion 21c in the state illustrated in FIG. 14.

When the first operation portion 400 in the state in FIG. 13A and FIG. 13B is operated and the first operation portion 400 moves to the fixation position, the operation restriction portion 400b moves, as illustrated in FIG. 14, to a position in which the operation restriction portion 400b can be in contact with the operation receiver 401w, and a movement of the lock member 401 is restricted. Therefore, a movement of the second operation portion 404 in the Yp direction is also restricted in this state, and it is not possible for a user to operate the second operation portion 404.

Thus, to detach the catheter unit 100 from the base unit 200, the first operation portion 400 is required to be first operated to the attachment-detachment position. In other words, the operation restriction portion 400b is disposed such that an operation of the second operation portion 404 is limited during a period in which an operation of moving the first operation portion 400 to the attachment-detachment position is not performed.

Consequently, even when a user attempts to detach the catheter unit 100 by mistake in a state in which the driving wire W is fixed to the coupling portion 21c, detachment of the catheter unit 100 is limited by the lock member 401.

In a state in which the catheter unit 100 is attached to the base unit 200 and in which the lock member 401 is in the lock position, the first operation portion 400 is movable between the fixation position and the attachment-detachment position. Thus, even when the first operation portion 400 is in the attachment-detachment position, detachment of the catheter unit 100 is limited when the lock member 401 is not moved to the unlock position. Therefore, a situation in which the catheter unit 100 becomes detachable from the base unit 200 when a user performs an operation of coupling or releasing the driving wire W to/from the coupling portion 21c by operating the first operation portion 400 is suppressed. In addition, a situation in which the catheter unit 100 is detached from the base unit 200 by mistake or unintentionally is suppressed.

Further, it is possible, without operating the first operation portion 400, to position the lock member 401 in the lock position and engage the lock member 401 with the engaged shape portion 25c by linearly attaching the catheter unit 100 to the base unit 200 in the attachment direction Da.

To detach the catheter unit 100 from the base unit 200, a user is required to operate the first operation portion 400 and the second operation portion 404 that are disposed at different positions (separated positions). The first operation portion 400 and the second operation portion 404 are movable independently from each other. Therefore, it is possible to reduce a possibility that, in an operation of one of the first operation portion 400 and the second operation portion 404, the other one of the first operation portion 400 and the second operation portion 404 is operated by mistake or unintentionally. In addition, to move one of the first operation portion 400 and the second operation portion 404, it is not necessary to move the other one of the first operation portion 400 and the second operation portion 404, and a load of a user can be reduced.

### Modifications

In the first operation portion 400 according to the present embodiment, the Rc direction is the coupling direction and the Rd direction is the release direction. the Rc direction and the Rd direction, however, may be the release direction and the coupling direction, respectively.

In the present embodiment, the catheter unit 100 includes the first operation portion 400 and the second operation portion 404. However, the base unit 200 may include one or both of the first operation portion 400 and the second operation portion 404. The base unit 200 also may include the lock member 401. In this case, a portion corresponding to the engaged shape portion 25c is included in the catheter unit 100.

In the present embodiment, locking by the lock member 401 is released by a movement of the second operation portion 404 in the Yp direction. The present invention is, however, not limited thereto. For example, locking by the lock member 401 may be released by using a second operation portion that moves in a direction (for example, the attachment-detachment direction DE) orthogonal to the Yf and Yp directions.

The present embodiment at least discloses the following matters.

### Configuration 1

A medical apparatus including:
a coupling unit that includes a coupling portion that is connected to a driving source;
a bending unit that is detachably attached to the coupling unit, the bending unit including a bending portion and a linear member that is connected to the bending portion;
a first operation portion that is movable to a fixation position and to an attachment-detachment position in a state in which the bending unit is attached to the coupling unit, the fixation position being a position in which the linear member is fixed to the coupling portion, the attachment-detachment position being a position in which the linear member is released from the coupling portion;
a lock member that is movable to a lock position and to an unlock position in a state in which the bending unit is attached to the coupling unit and in which the first operation portion is in the attachment-detachment position, the lock position being a position in which the lock member restricts detachment of the bending unit from the coupling unit; and
a second operation portion that moves the lock member from the lock position to the unlock position,
in which detachment of the bending unit from the coupling unit is allowed in a state in which the bending unit is attached to the coupling unit, in which the first operation portion is in the attachment-detachment position, and in which the lock member is in the unlock position, and
in which the first operation portion is movable between the fixation position and the attachment-detachment position in a state in which the bending unit is attached to the coupling unit and in which the lock member is in the lock position.

### Configuration 2

The medical apparatus according to the configuration 1,
in which the second operation portion is positioned away from the first operation portion.

### Configuration 3

A medical apparatus including:
a coupling unit that includes a coupling portion that is connected to a driving source;
a bending unit that is detachably attached to the coupling unit, the bending unit including a bending portion and a linear member that is connected to the bending portion;
a first operation portion that is movable to a fixation position and to an attachment-detachment position in a state in which the bending unit is attached to the coupling unit, the fixation position being a position in which the linear member is fixed to the coupling portion, the attachment-detachment position being a position in which the linear member is released from the coupling portion;
a lock member that is movable to a lock position and to an unlock position in a state in which the bending unit is attached to the coupling unit and in which the first operation portion is in the attachment-detachment position, the lock position being a position in which the lock member restricts detachment of the bending unit from the coupling unit; and
a second operation portion that moves the lock member from the lock position to the unlock position,
in which detachment of the bending unit from the coupling unit is allowed in a state in which the bending unit is attached to the coupling unit, in which the first operation portion is in the attachment-detachment position, and in which the lock member is in the unlock position, and
in which the second operation portion is positioned away from the first operation portion.

### Configuration 4

A medical apparatus including:
a coupling unit that includes a plurality of coupling portions, the plurality of coupling portions being each connected to a corresponding one of a plurality of driving sources;
a bending unit that is detachably attached to the coupling unit, the bending unit including a bending portion and a plurality of linear members that are connected to the bending portion;
a first operation portion that is movable to a fixation position and to an attachment-detachment position in a state in which the bending unit is attached to the coupling unit, the fixation position being a position in which each of the plurality of linear members is fixed to a corresponding one of the plurality of coupling portions, the attachment-detachment position being a position in which each of the plurality of linear members is released from the corresponding one of the plurality of coupling portions;
a lock member that is movable to a lock position and to an unlock position in a state in which the bending unit is attached to the coupling unit and in which the first operation portion is in the attachment-detachment position, the lock position being a position in which the lock member restricts detachment of the bending unit from the coupling unit; and
a second operation portion that moves the lock member from the lock position to the unlock position,
in which detachment of the bending unit from the coupling unit is allowed in a state in which the bending unit is attached to the coupling unit, in which the first operation portion is in the attachment-detachment position, and in which the lock member is in the unlock position, and
in which the first operation portion is movable between the fixation position and the attachment-detachment position in a state in which the bending unit is attached to the coupling unit and in which the lock member is in the lock position.

### Configuration 5

The medical apparatus according to the configuration 4,
in which the second operation portion is positioned away from the first operation portion.

### Configuration 6

A medical apparatus including:
a coupling unit that includes a plurality of coupling portions, the plurality of coupling portions being each connected to a corresponding one of a plurality of driving sources;
a bending unit that is detachably attached to the coupling unit, the bending unit including a bending portion and a plurality of linear members that are connected to the bending portion;
a first operation portion that is movable to a fixation position and to an attachment-detachment position in a state in which the bending unit is attached to the coupling unit, the fixation position being a position in which each of the plurality of linear members is fixed to a corresponding one of the plurality of coupling portions, the attachment-detachment position being a position in which each of the plurality of linear members is released from the corresponding one of the plurality of coupling portions;
a lock member that is movable to a lock position and to an unlock position in a state in which the bending unit is attached to the coupling unit and in which the first operation portion is in the attachment-detachment position, the lock position being a position in which the lock member restricts detachment of the bending unit from the coupling unit; and
a second operation portion that moves the lock member from the lock position to the unlock position,
in which detachment of the bending unit from the coupling unit is allowed in a state in which the bending unit is attached to the coupling unit, in which the first operation portion is in the attachment-detachment position, and in which the lock member is in the unlock position, and
in which the second operation portion is positioned away from the first operation portion.

### Configuration 7

The medical apparatus according to any one of the configurations 1 to 6,
in which a movement of the lock member from the lock position to the unlock position is restricted in a state in which the bending unit is attached to the coupling unit and in which the first operation portion is in the fixation position.

### Configuration 8

The medical apparatus according to any one of the configurations 1 to 7,
in which an operation of the second operation portion that moves the lock member from the lock position to the unlock position is restricted in a state in which the bending unit is attached to the coupling unit and in which the first operation portion is in the fixation position.

### Configuration 9

The medical apparatus according to any one of the configurations a to 8,
in which the first operation portion is rotatable about a rotation axis extending in an attachment-detachment direction of the bending unit.

### Configuration 10

The medical apparatus according to the configuration 9,
in which the second operation portion is movable in a direction intersecting an extension direction of the rotation axis.

### Configuration 11

The medical apparatus according to the configuration 9 or 10,
in which the second operation portion is movable in a rotation radius direction of the first operation portion.

### Configuration 12

The medical apparatus according to any one of the configurations 1 to 11,
in which, in attaching the bending unit to the coupling unit, the lock member is moved in a direction from the lock position toward the unlock position.

### Configuration 13

The medical apparatus according to any one of the configurations 1 to 12, including:
an urging member that urges the lock member in a direction from the unlock position toward the lock position.

### Configuration 14

The medical apparatus according to any one of the configurations 1 to 13,
in which the lock member is swingable between the unlock position and the lock position.

### Configuration 15

The medical apparatus according to any one of the configurations 1 to 14,
in which the second operation portion is included in the bending unit.

The present invention is not limited to the aforementioned embodiments and can be variously changed and modified without deviating from the spirit and the scope of the present invention. Accordingly, the following claims are attached to publish the scope of the present invention.

As described above, according to the present invention, it is possible to suppress a situation in which an operation for releasing a linear member from a coupling portion causes a bending unit to be detachable from a coupling unit.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. A medical apparatus comprising:
a coupling unit that includes a plurality of coupling portions, the plurality of coupling portions being each connected to a corresponding one of a plurality of driving sources;
a bending unit that is detachably attached to the coupling unit, the bending unit including a bending portion and a plurality of linear members that are connected to the bending portion;
a first operation portion that is movable to a fixation position and to an attachment-detachment position in a state in which the bending unit is attached to the coupling unit, the fixation position being a position in which each of the plurality of linear members is fixed to a corresponding one of the plurality of coupling portions, the attachment-detachment position being a position in which each of the plurality of linear members is released from the corresponding one of the plurality of coupling portions;
a lock member that is movable to a lock position and to an unlock position in a state in which the bending unit is attached to the coupling unit and in which the first operation portion is in the attachment-detachment position, the lock position being a position in which the lock member restricts detachment of the bending unit from the coupling unit; and
a second operation portion that moves the lock member from the lock position to the unlock position,
wherein detachment of the bending unit from the coupling unit is allowed in a state in which the bending unit is attached to the coupling unit, in which the first operation portion is in the attachment-detachment position, and in which the lock member is in the unlock position, and
wherein the first operation portion is movable between the fixation position and the attachment-detachment position in a state in which the bending unit is attached to the coupling unit and in which the lock member is in the lock position.

2. The medical apparatus according to claim 1,
wherein the second operation portion is positioned away from the first operation portion.

3. A medical apparatus comprising:
a coupling unit that includes a plurality of coupling portions, the plurality of coupling portions being each connected to a corresponding one of a plurality of driving sources;
a bending unit that is detachably attached to the coupling unit, the bending unit including a bending portion and a plurality of linear members that are connected to the bending portion;
a first operation portion that is movable to a fixation position and to an attachment-detachment position in a state in which the bending unit is attached to the coupling unit, the fixation position being a position in which each of the plurality of linear members is fixed to a corresponding one of the plurality of coupling portions, the attachment-detachment position being a position in which each of the plurality of linear members is released from the corresponding one of the plurality of coupling portions;
a lock member that is movable to a lock position and to an unlock position in a state in which the bending unit is attached to the coupling unit and in which the first operation portion is in the attachment-detachment position, the lock position being a position in which the lock member restricts detachment of the bending unit from the coupling unit; and
a second operation portion that moves the lock member from the lock position to the unlock position,
wherein detachment of the bending unit from the coupling unit is allowed in a state in which the bending unit is attached to the coupling unit, in which the first operation portion is in the attachment-detachment position, and in which the lock member is in the unlock position, and
wherein the second operation portion is positioned away from the first operation portion.

4. The medical apparatus according to claim 1,
wherein a movement of the lock member from the lock position to the unlock position is restricted in a state in which the bending unit is attached to the coupling unit and in which the first operation portion is in the fixation position.

5. The medical apparatus according to claim 1,
wherein an operation of the second operation portion that moves the lock member from the lock position to the unlock position is restricted in a state in which the bending unit is attached to the coupling unit and in which the first operation portion is in the fixation position.

6. The medical apparatus according to claim 1,
wherein the first operation portion is rotatable about a rotation axis extending in an attachment-detachment direction of the bending unit.

7. The medical apparatus according to claim 6,
wherein the second operation portion is movable in a direction intersecting an direction of the rotation axis.

8. The medical apparatus according to claim 6,
wherein the second operation portion is movable in a rotation radius direction of the first operation portion.

9. The medical apparatus according to claim 1,
wherein, in attaching the bending unit to the coupling unit, the lock member is moved in a direction from the lock position toward the unlock position.

10. The medical apparatus according to claim 1, comprising:
an urging member that urges the lock member in a direction from the unlock position toward the lock position.

11. The medical apparatus according to claim 1,
wherein the lock member is swingable between the unlock position and the lock position.

12. The medical apparatus according to claim 1,
wherein the second operation portion is included in the bending unit.
